(19) United States
(12) Patent Application Publication
(10) Pub. No.: US 2026/0252806 A1
(43) Pub. Date: Aug. 27, 2026

(54) SYSTEM

(71) Applicant: SoftBank Group Corp., Tokyo (JP)

(72) Inventor: Yoshikazu OKADA, Tokyo (JP)

(73) Assignee: SoftBank Group Corp., Tokyo (JP)

(21) Appl. No.: 19/536,306

(22) Filed: Feb. 11, 2026

(30) Foreign Application Priority Data
Feb. 21, 2025 (JP) 2025-027087

(51) Int. Cl. G06F 40/30; G06Q 30/02

(52) U.S. Cl. CPC G06F 40/30; G06Q 30/02

(57) ABSTRACT

The system according to the embodiment comprises a registration unit, a collection unit, an analysis unit, a suggestion unit, and a modification unit. The registration unit registers a user's target weight or deadline. The collection unit collects data such as the user's weight, exercise data, and photographs of food consumed. The analysis unit analyzes the data collected by the collection unit. The suggestion unit performs meal planning, recipe creation, and exercise plan creation based on the analysis results obtained by the analysis unit. The modification unit modifies the content proposed by the suggestion unit.

### CROSS-REFERENCE TO RELATED APPLICATION

The present application claims priority to and incorporates by reference the entire contents of Japanese Patent Application No. 2025-027087 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it has been difficult for people on a diet to plan appropriate meals or exercise plans based on their target weight or deadline, and there is room for improvement.

### SUMMARY OF THE INVENTION

The system according to the embodiment comprises a registration unit, a collection unit, an analysis unit, a suggestion unit, and a modification unit. The registration unit registers a user's target weight or deadline. The collection unit collects data such as the user's weight, exercise data, and photographs of food consumed. The analysis unit analyzes the data collected by the collection unit. The suggestion unit performs meal planning, recipe creation, and exercise plan creation based on the analysis results obtained by the analysis unit. The modification unit modifies the content proposed by the suggestion unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

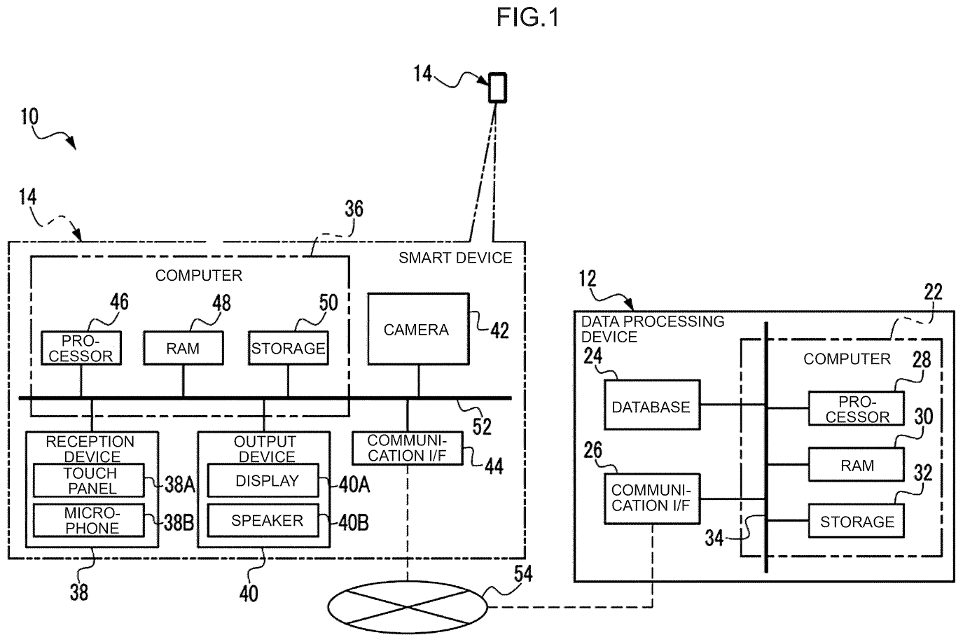

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

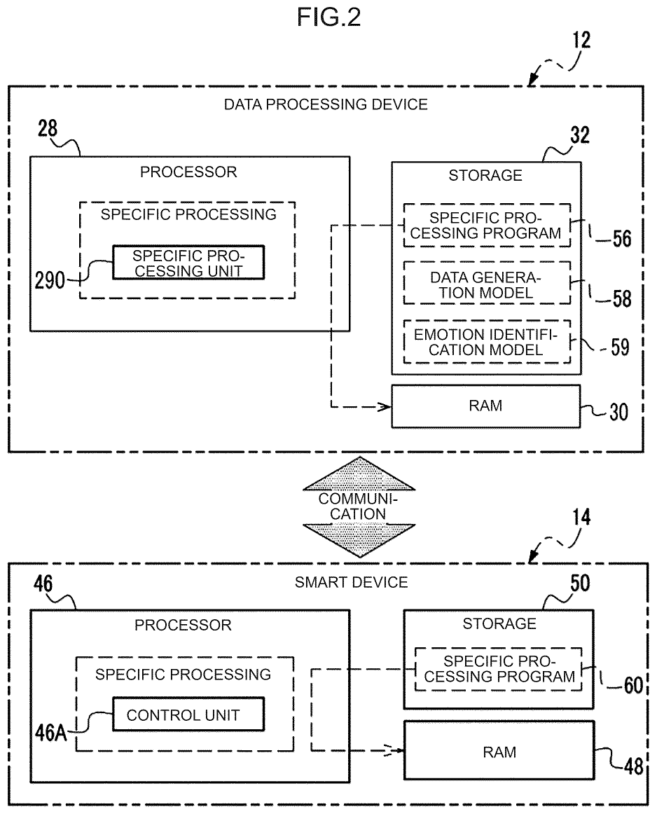

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The diet support system according to the embodiment of the present invention is a system in which a user registers a target weight or deadline, and based on that, automatically performs “meal planning,” “recipe creation,” and “exercise plan creation.” This diet support system modifies and proposes “meal planning,” “recipe creation,” and “exercise plan creation” each time the user inputs information such as weight, exercise data, or photographs of food consumed. For example, when a user sets a goal such as “I want to lose 5 kg in 3 months,” this information is input into the system. Next, the system performs optimal “meal planning,” “recipe creation,” and “exercise plan creation” based on the user's goal. For example, it proposes meal plans considering calorie restrictions, recipes with balanced nutrition, and effective exercise plans. Each time the user inputs weight, exercise data, or photographs of food consumed, the system analyzes this information and modifies the proposed content. For example, when the user inputs a photograph of food consumed, the system analyzes the calories and nutrients and reflects them in the next meal plan or recipe. It can also adjust the exercise plan based on exercise data. With this mechanism, the user can always receive an optimal diet plan tailored to their goal. For example, if the user is approaching the target weight, the system can propose increasing the amount of exercise. Conversely, if the user is moving away from the target weight, the system can propose strengthening calorie restrictions. This system effectively supports the user's diet and helps achieve their goals. Thus, the diet support system can automatically propose and modify diet plans based on the user's target weight or deadline. Specifically, this diet support system is composed of multiple computer modules (registration unit, collection unit, analysis unit, suggestion unit, modification unit), each operating on a dedicated processor. The registration unit receives the user's input target weight (e.g., 60 kg→55 kg) and deadline (e.g., 90 days) as numerical data and stores them in a database. The collection unit acquires weight data entered by the user via a smartphone app or wearable device (e.g., daily weight records in kg), exercise data (e.g., step count, calories burned, exercise time, heart rate as time-series vectors), and meal images (e.g., RGB images, resolution 1280×720 pixels), and performs preprocessing (noise removal, normalization, feature extraction). The analysis unit uses the collected data as input, estimates ingredients and dish names from images using convolutional neural networks (CNN) or transformer models, and calculates nutrient amounts such as calories, protein, fat, and carbohydrates using a nutrient estimation model (e.g., multilayer perceptron). For exercise data, it extracts activity patterns and estimates calories burned and exercise intensity using recurrent neural networks (RNN) or time-series analysis algorithms. Examples of AI input include (1) meal image tensor (3×1280×720), (2) time-series array of weight (e.g., real-value vector for 90 days), (3) multidimensional vector of exercise data (e.g., time-series arrays of step count, heart rate, calories burned). Examples of AI output include (1) dish label from meal image (e.g., “Grilled chicken breast”), (2) estimated nutrient values (e.g., 350 kcal, 30 g protein), (3) exercise intensity score (e.g., 1.2 METs), (4) diet progress score (e.g., goal achievement rate 65%). The suggestion unit combines rule-based and reinforcement learning algorithms based on the output results of the analysis unit to generate optimal meal plans (e.g., 1500 kcal per day, combinations of main dish, side dish, staple food), recipes (e.g., cooking steps, quantities, cooking time), and exercise plans (e.g., 30 minutes of aerobic exercise plus strength training three times a week) for goal achievement. The modification unit re-inputs data into the AI model each time new data is entered by the user and modifies the plan to reflect the latest progress and trends. For example, if the user is approaching the target weight, it automatically generates proposals to increase exercise; if moving away, it generates proposals to strengthen dietary restrictions. This series of processing is technically characterized by autonomous feature extraction, pattern recognition, and optimization in high-dimensional data space by AI, unlike conventional manual work or simple rule-based processing. For AI model training, error backpropagation using a loss function (e.g., squared error of the difference from the target weight) and data augmentation (e.g., image rotation, scaling) are used to improve generalization performance. Subsequent processing utilizes AI output for threshold judgment (e.g., plan enhancement if goal achievement rate is less than 80%) and branching (e.g., prioritizing exercise or meal plan), realizing optimized diet support for each user. As a technical effect, this system can analyze large and diverse data in real time and automatically generate and modify individually optimized plans, achieving significant improvement in proposal accuracy, increased user retention, and overall system operational efficiency (reduction of manual intervention) compared to conventional systems. Specific application fields include personal diet apps, corporate health management support services, and lifestyle disease prevention programs in medical institutions.

The diet support system according to the embodiment comprises a registration unit, a collection unit, an analysis unit, a suggestion unit, and a modification unit. The registration unit registers the user's target weight or deadline. For example, the user sets a goal such as “I want to lose 5 kg in 3 months.” This information is input into the system. The collection unit collects data such as the user's weight, exercise data, and photographs of food consumed. For example, the collection unit can collect photographs of food consumed taken by the user using a smartphone camera. The collection unit can also cooperate with a wearable device to collect exercise data. The analysis unit analyzes the data collected by the collection unit. For example, the analysis unit can analyze the collected data using a machine learning algorithm. The suggestion unit performs “meal planning,” “recipe creation,” and “exercise plan creation” based on the analysis results obtained by the analysis unit. For example, the suggestion unit proposes meal plans considering calorie restrictions, recipes with balanced nutrition, and effective exercise plans. The modification unit modifies the content proposed by the suggestion unit. For example, each time the user inputs weight, exercise data, or photographs of food consumed, the modification unit analyzes this information and modifies the proposed content. Thus, the diet support system according to the embodiment can automatically propose and modify diet plans based on the user's target weight or deadline. Specifically, this diet support system has a clear computer module configuration of registration unit, collection unit, analysis unit, suggestion unit, and modification unit, each operating on a dedicated processor. The registration unit receives the user's input target weight (e.g., 60 kg→55 kg) and deadline (e.g., 90 days) as numerical data and stores them in a database. The collection unit acquires weight data entered by the user via a smartphone app or wearable device (e.g., daily weight records in kg), exercise data (e.g., step count, calories burned, exercise time, heart rate as time-series vectors), and meal images (e.g., RGB images, resolution 1280×720 pixels), and performs preprocessing (noise removal, normalization, feature extraction). The analysis unit uses the collected data as input, estimates ingredients and dish names from images using convolutional neural networks (CNN) or transformer models, and calculates nutrient amounts such as calories, protein, fat, and carbohydrates using a nutrient estimation model (e.g., multilayer perceptron). For exercise data, it extracts activity patterns and estimates calories burned and exercise intensity using recurrent neural networks (RNN) or time-series analysis algorithms. Examples of AI input include (1) meal image tensor (3×1280×720), (2) time-series array of weight (e.g., real-value vector for 90 days), (3) multidimensional vector of exercise data (e.g., time-series arrays of step count, heart rate, calories burned). Examples of AI output include (1) dish label from meal image (e.g., “Grilled chicken breast”), (2) estimated nutrient values (e.g., 350 kcal, 30 g protein), (3) exercise intensity score (e.g., 1.2 METs), (4) diet progress score (e.g., goal achievement rate 65%). The suggestion unit combines rule-based and reinforcement learning algorithms based on the output results of the analysis unit to generate optimal meal plans (e.g., 1500 kcal per day, combinations of main dish, side dish, staple food), recipes (e.g., cooking steps, quantities, cooking time), and exercise plans (e.g., 30 minutes of aerobic exercise plus strength training three times a week) for goal achievement. The modification unit re-inputs data into the AI model each time new data is entered by the user and modifies the plan to reflect the latest progress and trends. For example, if the user is approaching the target weight, it automatically generates proposals to increase exercise; if moving away, it generates proposals to strengthen dietary restrictions. This series of processing is technically characterized by autonomous feature extraction, pattern recognition, and optimization in high-dimensional data space by AI, unlike conventional manual work or simple rule-based processing. For AI model training, error backpropagation using a loss function (e.g., squared error of the difference from the target weight) and data augmentation (e.g., image rotation, scaling) are used to improve generalization performance. Subsequent processing utilizes AI output for threshold judgment (e.g., plan enhancement if goal achievement rate is less than 80%) and branching (e.g., prioritizing exercise or meal plan), realizing optimized diet support for each user. As a technical effect, this system can analyze large and diverse data in real time and automatically generate and modify individually optimized plans, achieving significant improvement in proposal accuracy, increased user retention, and overall system operational efficiency (reduction of manual intervention) compared to conventional systems. Specific application fields include personal diet apps, corporate health management support services, and lifestyle disease prevention programs in medical institutions.

The collection unit comprises an image recognition unit for analyzing photographs of food consumed. The image recognition unit, for example, analyzes photographs of food consumed. For example, the image recognition unit uses object detection algorithms to identify the types of food consumed and analyze calories and nutrients. The image recognition unit can also classify photographs of food consumed using image classification algorithms to accurately grasp the meal content. Thus, by analyzing photographs of food consumed, the meal content can be accurately grasped. Specifically, the image recognition unit uses deep learning architectures such as convolutional neural networks (CNN) and transformer-based image recognition models to extract multiple feature maps from meal images. The image recognition unit receives an RGB image tensor (e.g., 3×1280×720 pixels) as input and performs preprocessing such as resizing, noise removal, color space conversion, and histogram normalization. Next, the image recognition unit extracts low-level features such as edges, textures, and color distributions in the convolutional layers of the CNN, and further extracts high-level features such as ingredient shapes and plating patterns in the deeper layers. The image recognition unit uses object detection heads (e.g., YOLO, Faster R-CNN) to detect multiple ingredient regions in the image as bounding boxes, and for each region, uses classification heads to estimate dish names and ingredient labels (e.g., “white rice,” “grilled salmon,” “miso soup”). The image recognition unit inputs the estimated label information into a nutrient estimation model (e.g., multilayer perceptron) to calculate the amount of nutrients such as calories, protein, fat, and carbohydrates for each dish or ingredient. Examples of AI input include (1) meal image tensor (3×1280×720), (2) coordinates vector of detected regions in the image (e.g., N bounding box coordinates), (3) image feature vector (e.g., 2048 dimensions). Examples of AI output include (1) dish label (e.g., “Grilled chicken breast”), (2) ingredient label (e.g., “broccoli”), (3) estimated nutrient values (e.g., 350 kcal, 30 g protein). The image recognition unit outputs the estimation results as structured data to subsequent units such as the suggestion unit and modification unit, and utilizes them for subsequent processing such as meal planning, recipe creation, and meal history management. Unlike conventional manual visual confirmation or simple image comparison, the image recognition unit autonomously performs feature extraction and pattern recognition in high-dimensional space, greatly improving the accuracy of automatic meal content recognition. As a technical effect, the image recognition unit realizes highly accurate dish and ingredient recognition and nutrient estimation for diverse meal images, enabling automation and efficiency of meal management and health support for each user. Specific application fields include personal meal recording apps, nutrition guidance support in medical institutions, and corporate health management platforms.

The collection unit comprises a cooperation unit for collecting exercise data in cooperation with a wearable device. The cooperation unit, for example, collects exercise data in cooperation with a wearable device. For example, the cooperation unit can collect exercise data such as step count, calories burned, and exercise time in cooperation with a smartwatch or fitness tracker. Thus, cooperation with a wearable device enables accurate collection of exercise data. Specifically, the cooperation unit uses Bluetooth Low Energy (BLE) or Wi-Fi communication protocols to acquire exercise data in real time from wearable devices such as smartwatches and fitness trackers. The cooperation unit receives multidimensional data transmitted from the device, such as step count (e.g., 1,000-20,000 steps per day), calories burned (e.g., 1,500 kcal per day), exercise time (e.g., in 30-minute units), heart rate (e.g., time-series vector at 1-minute intervals), and exercise type (e.g., category labels such as running, walking, cycling). The cooperation unit performs preprocessing such as timestamping, missing value imputation, outlier removal, and unit conversion on the received data, and stores it in a standardized time-series database. The cooperation unit dynamically adjusts the data collection frequency and communication interval according to the user's activity status and battery level, optimizing communication load and power consumption. Examples of AI input include (1) time-series array of step count (e.g., integer vector per day), (2) time-series vector of heart rate (e.g., real-value array per minute), (3) category label array of exercise type (e.g., exercise type for 7 days). Examples of AI output include (1) exercise intensity score (e.g., 1.2 METs), (2) estimated calories burned (e.g., 1,800 kcal), (3) exercise pattern classification label (e.g., “aerobic exercise-focused,” “strength training-focused”). The cooperation unit transmits the AI output results to the suggestion unit and modification unit for generating individually optimized exercise plans and progress management. Unlike conventional manual input or simple aggregation processing, the cooperation unit collects and integrates diverse sensor data in real time and with high accuracy, greatly improving the comprehensiveness and accuracy of exercise data. As a technical effect, the cooperation unit automatically accumulates and analyzes exercise history for each user, realizing advanced and efficient health management and diet support. Specific application fields include personal fitness apps, corporate health management support, and rehabilitation management in medical institutions.

The collection unit comprises a security unit for performing encryption or anonymization of data. The security unit, for example, performs encryption or anonymization of data. For example, the security unit can encrypt data using AES or RSA encryption. The security unit can also perform data masking or pseudonymization. Thus, encryption or anonymization of data enables protection of user privacy. Specifically, the security unit applies encryption or anonymization processing to personal information such as weight data, exercise data, and meal image data collected from users before storing it in the database. The security unit uses symmetric key encryption (e.g., AES-256) or public key encryption (e.g., RSA-2048) to encrypt data at the binary level and strictly manages decryption keys. The security unit ensures data anonymity by hashing or tokenizing personal identifiers (e.g., name, email address, device ID). Furthermore, the security unit automatically masks facial regions and deletes metadata in image data to reduce the risk of personal identification from images. The security unit implements user authentication (e.g., password, one-time token) and access rights management during data access to prevent unauthorized access and information leakage. Examples of AI input include (1) encrypted data blocks, (2) anonymized data sets, (3) access log vectors (e.g., access date and time, device type, operation type). Examples of AI output include (1) abnormal access detection label (e.g., “suspected unauthorized access”), (2) data decryption availability flag, (3) anonymization level score (e.g., 0.95). The security unit automatically executes access control and additional security enhancement processing based on AI output results. Unlike conventional simple password protection or manual masking, the security unit autonomously combines multi-layered encryption, anonymization, and access control, greatly improving the strength and operational efficiency of data protection. As a technical effect, the security unit realizes advanced protection of user privacy and compliance with legal regulations, providing a secure foundation for diet support that enables safe data utilization. Specific application fields include personal health management apps, electronic medical record systems in medical institutions, and corporate health management platforms.

The collection unit collects photographs of food consumed using a smartphone camera. The collection unit, for example, collects photographs of food consumed using a smartphone camera. For example, the collection unit can take photographs of food consumed with a smartphone camera and collect the data. Thus, using a smartphone camera enables easy collection of meal content. Specifically, the collection unit uses the smartphone camera API to acquire image data (e.g., JPEG format, resolution 1280×720 pixels) taken by the user before and after meals in real time. The collection unit simultaneously records metadata such as timestamp, location information (GPS coordinates), and device ID at the time of image acquisition and stores them in the meal history database. The collection unit automatically performs preprocessing such as resizing, noise removal, and color correction on the image data to optimize it as input for the image recognition unit. Examples of AI input include (1) meal image tensor (3×1280×720), (2) vector of shooting time and location information (e.g., 2024-06-01 12:00, latitude 35.6, longitude 139.7), (3) device ID label (e.g., “device_12345”). Examples of AI output include (1) dish label (e.g., “salad chicken”), (2) ingredient label (e.g., “tomato”), (3) image quality score (e.g., 0.98). The collection unit utilizes AI output results for subsequent processing such as automatic recording of meal history, meal planning proposals, and nutrient estimation. Unlike conventional manual input or text recording, the collection unit directly acquires and utilizes image data, greatly improving the accuracy and convenience of meal content recording. As a technical effect, the collection unit minimizes user burden and realizes automation and high accuracy of meal management. Specific application fields include personal meal recording apps, nutrition guidance in medical institutions, and corporate health management support services.

The analysis unit analyzes the collected data using a machine learning algorithm. The analysis unit, for example, analyzes the collected data using a machine learning algorithm. For example, the analysis unit can analyze data using deep learning or support vector machines. Thus, using machine learning algorithms improves the accuracy of data analysis. Specifically, the analysis unit receives diverse data as input from the collection unit, such as weight data (e.g., real-value vector for 90 days), exercise data (e.g., time-series arrays of step count, heart rate, calories burned), and meal image data (e.g., 3×1280×720 tensor). For weight and exercise data, the analysis unit uses recurrent neural networks (RNN) and time-series analysis algorithms (e.g., LSTM, GRU) to extract activity patterns and weight fluctuation trends. For meal image data, the analysis unit uses convolutional neural networks (CNN) and transformer models to estimate dish and ingredient labels and nutrient amounts from images. The analysis unit can also use machine learning models such as support vector machines (SVM) and decision trees to classify and perform regression analysis on the user's diet progress and risk scores. Examples of AI input include (1) time-series vector of weight (e.g., 90 days), (2) multidimensional array of exercise data (e.g., step count, heart rate, calories burned), (3) meal image tensor (3×1280×720). Examples of AI output include (1) diet progress score (e.g., goal achievement rate 65%), (2) risk classification label (e.g., “plateau,” “smooth progress”), (3) estimated nutrient values (e.g., 350 kcal, 30 g protein). The analysis unit transmits AI output results to the suggestion unit and modification unit for generating individually optimized meal and exercise plans and progress management. Unlike conventional simple statistical processing or manual analysis, the analysis unit autonomously performs feature extraction, pattern recognition, and optimization in high-dimensional data space, greatly improving the accuracy and efficiency of data analysis. As a technical effect, the analysis unit integratively analyzes diverse data and realizes automatic generation of individually optimized health support and diet plans for each user. Specific application fields include personal health management apps, lifestyle disease prevention in medical institutions, and corporate health management support services.

The registration unit can estimate a user's emotion and adjust the setting of the target weight or deadline based on the estimated emotion. The registration unit, for example, estimates a user's emotion and adjusts the setting of the target weight or deadline based on the estimated emotion. For example, if the user is feeling stressed, the registration unit relaxes the setting of the target weight or deadline. If the user is highly motivated, the registration unit can set stricter target weight or deadline. Furthermore, if the user is feeling anxious, the registration unit can set the target weight or deadline in stages. Thus, by adjusting the setting of the target weight or deadline according to the user's emotion, more realistic goal setting becomes possible. Emotion estimation is realized, for example, by using an emotion engine or generative AI with emotion estimation functionality. Generative AI may be a text generation AI (e.g., LLM) or multimodal generative AI, but is not limited to these examples. Specifically, the registration unit inputs text data obtained from the user (e.g., diary entries, meal record comments, questionnaire responses as natural language text, length 50-500 tokens), audio data (e.g., 1-minute audio waveform, 16 kHz sampling, PCM format), and image data (e.g., facial images, expression images, 3×224×224 pixels) into an emotion estimation AI model. The registration unit performs preprocessing on the input data (e.g., tokenization of text, spectrogram conversion of audio, facial region extraction from images) and generates feature vectors optimized for the AI model (e.g., text embedding vectors, audio feature vectors, image feature vectors). The registration unit uses a multimodal emotion estimation model combining transformer-based large language models (e.g., 12 layers, hidden layer 768 dimensions), convolutional neural networks (e.g., ResNet-18), and recurrent neural networks for audio emotion classification (e.g., 2-layer LSTM). The registration unit obtains emotion labels (e.g., “stress,” “high motivation,” “anxiety”), emotion scores (e.g., stress level 0.72, motivation level 0.85), and emotion change trends (e.g., transition graph for the past 7 days) as AI model output. For example, if input such as “Recently, dieting is tough” text, a smiling facial image, and calm voice audio is provided, the AI outputs scores such as “stress level 0.65,” “anxiety level 0.40,” “motivation level 0.30.” In another example, if input such as “I worked hard on exercise today!” text, energetic voice, and bright facial image is provided, the output is “motivation level 0.90,” “stress level 0.10.” The registration unit uses these emotion scores for threshold judgment (e.g., relax goals if stress level is 0.6 or higher, strengthen goals if motivation level is 0.8 or higher) and rule-based branching to automatically adjust the target weight or deadline. Furthermore, the registration unit records the user's emotion change history as a time series and utilizes it as learning data for goal setting history management and future goal adjustment algorithms. Unlike conventional subjective counseling or simple questionnaire aggregation by humans, the registration unit combines multimodal emotion estimation in high-dimensional feature space and rule-based automatic adjustment to realize individually optimized and realistic goal setting for each user. For AI model training, supervised learning using large-scale emotion-annotated datasets, error backpropagation using loss functions such as cross-entropy loss and mean squared error, and data augmentation (e.g., text paraphrase generation, audio pitch conversion, image rotation) are used to improve model generalization and robustness. As a technical effect, the registration unit can grasp the user's emotional state in real time and with high accuracy, greatly improving the realism and achievability of goal setting, thereby enhancing diet continuation rate, goal achievement rate, and overall user experience of the system. Specific application fields include personal diet apps, lifestyle disease prevention programs in medical institutions, corporate health management support services, and mental health care support systems.

The registration unit can analyze the user's past diet history and propose an optimal target setting method. The registration unit, for example, analyzes the user's past diet history and proposes an optimal target setting method. For example, the registration unit sets goals by referring to diet methods that the user has succeeded with in the past. The registration unit can also set goals to avoid diet methods that the user has failed with in the past. Furthermore, the registration unit can propose an optimal goal achievement period based on the user's past diet history. Thus, by analyzing past diet history, optimal goal setting for the user becomes possible. Specifically, the registration unit obtains diet history data recorded as a time series for each user (e.g., weight transition vector for the past 5 years, start and end dates of each diet period, target weight, actual weight, achievement status, labels of diet methods used, weekly exercise amount, meal content, health records as structured data) from the database. The registration unit performs feature extraction (e.g., goal achievement rate, average weight loss speed, rebound occurrence, success rate for each diet method, labeling of failure factors) on these history data and uses machine learning models (e.g., decision trees, random forests, gradient boosting, clustering algorithms) and rule-based algorithms to automatically analyze the user's past success and failure patterns. Examples of AI input include (1) time-series vector of weight transition (e.g., 365 days of real values), (2) category label array of diet methods (e.g., “low-carb,” “aerobic exercise,” “fasting”), (3) structured data of each period's goals, achievements, and achievement status (e.g., JSON format). Examples of AI output include (1) recommended target setting method label (e.g., “gradual weight loss,” “short-term intensive”), (2) recommended goal achievement period (e.g., 90 days, 180 days), (3) recommended diet method (e.g., “aerobic exercise +balanced diet”), (4) failure risk score (e.g., 0.25). For example, if there are many successful histories with “low-carb+aerobic exercise,” the same method is recommended, and if there are many failures with “fasting,” that method is excluded. Furthermore, the registration unit uses AI output results for subsequent processing such as automatic initial input of goal setting screen, generation of goal setting advice messages for the user, and automatic adjustment of goal achievement period. Unlike conventional subjective counseling or simple history reference by humans, the registration unit autonomously performs feature extraction and pattern recognition of high-dimensional history data by AI, realizing automatic proposal of individually optimized target setting methods for each user. For AI model training, classification and regression tasks using past diet history and goal achievement status as training data, error backpropagation using loss functions such as cross-entropy loss and mean squared error, and data augmentation (e.g., time-series shift of history data, sampling of method labels) are used to improve model generalization. As a technical effect, the registration unit analyzes the user's past behavior and achievement data with high accuracy and automates individually optimized and realistic goal setting, thereby improving goal achievement rate, preventing rebound, enhancing user satisfaction, and improving system operational efficiency. Specific application fields include personal diet apps, lifestyle disease prevention programs in medical institutions, corporate health management support services, and personal training support systems.

The registration unit can perform filtering based on the user's health status or lifestyle habits when setting the target weight or deadline. The registration unit, for example, performs filtering based on the user's health status or lifestyle habits when setting the target weight or deadline. For example, the registration unit sets the target weight by referring to the user's health checkup results. The registration unit can also set the target deadline by considering the user's lifestyle habits (diet, exercise, sleep). Furthermore, the registration unit can set reasonable goals by considering the user's medical history. Thus, by setting goals based on the user's health status or lifestyle habits, a reasonable diet becomes possible. Specifically, the registration unit obtains health checkup data from the user (e.g., height, weight, BMI, blood pressure, blood glucose, cholesterol, liver function values as numerical vectors), lifestyle habit data (e.g., one week of meal records, exercise frequency, average sleep time, presence or absence of drinking/smoking habits), and medical history data (e.g., past disease labels, treatment history, medication information) from the database and inputs them as structured data into the AI model. The registration unit performs preprocessing (e.g., missing value imputation, normalization, category conversion), feature extraction (e.g., health risk score, lifestyle habit score, disease risk label), and uses machine learning models (e.g., random forest, logistic regression, neural network) and rule-based algorithms to perform filtering of target weight and deadline according to the user's health status and lifestyle habits. Examples of AI input include (1) health checkup numerical vector (e.g., 10 dimensions), (2) lifestyle habit category label array (e.g., “skip breakfast,” “exercise twice a week,” “6 hours sleep”), (3) medical history label array (e.g., “hypertension,” “diabetes”). Examples of AI output include (1) recommended target weight range (e.g., 55-58 kg), (2) recommended target deadline (e.g., 120 days), (3) risk warning label (e.g., “rapid weight loss not allowed”), (4) recommended diet method (e.g., “balanced diet +light exercise”). For example, if hypertension is found in a health checkup, the system automatically sets goals to avoid rapid weight loss or high-intensity exercise, and if there is a tendency for sleep deprivation, it proposes goals that prioritize improving lifestyle rhythm. Furthermore, the registration unit uses AI output results for subsequent processing such as input restrictions on the goal setting screen (e.g., automatic blocking of dangerous goal values), health risk warning display to the user, and automatic adjustment of goal achievement plans. Unlike conventional subjective advice or simple goal input by humans, the registration unit combines integrated analysis of multidimensional health and lifestyle habit data with rule-based automatic filtering to realize safe and reasonable goal setting for each user. For AI model training, classification and regression tasks using health checkup data and diet achievement data as training data, error backpropagation using loss functions such as cross-entropy loss and mean squared error, and data augmentation (e.g., adding noise to health checkup values, sampling lifestyle habit patterns) are used to improve model generalization. As a technical effect, the registration unit automatically avoids health risks and realizes high-precision, realistic, and safe goal setting, thereby reducing health risk, improving goal achievement rate, enhancing user satisfaction, and improving system operational efficiency. Specific application fields include personal diet apps, lifestyle disease prevention programs in medical institutions, corporate health management support services, and health promotion programs by insurance companies.

The registration unit can estimate a user's emotion and determine the priority of target setting based on the estimated emotion. The registration unit, for example, estimates a user's emotion and determines the priority of target setting based on the estimated emotion. For example, if the user is highly motivated, the registration unit prioritizes short-term goals. If the user is feeling anxious, the registration unit can prioritize long-term goals. Furthermore, if the user is feeling stressed, the registration unit can prioritize realistic goals. Thus, by determining the priority of target setting according to the user's emotion, more effective dieting becomes possible. Emotion estimation is realized, for example, by using an emotion engine or generative AI with emotion estimation functionality. Generative AI may be a text generation AI (e.g., LLM) or multimodal generative AI, but is not limited to these examples. Specifically, the registration unit inputs natural language text obtained from the user (e.g., comments when entering diet goals, diary entries, questionnaire responses), audio data (e.g., 1-minute audio waveform), and image data (e.g., facial expression images) into an emotion estimation AI model. The registration unit performs preprocessing on the input data (e.g., tokenization of text, spectrogram conversion of audio, facial region extraction from images) and generates feature vectors. The registration unit uses a multimodal emotion estimation model combining transformer-based large language models, convolutional neural networks, and recurrent neural networks for audio emotion classification. The registration unit obtains emotion labels (e.g., “high motivation,” “anxiety,” “stress”) and emotion scores (e.g., motivation level 0.85, anxiety level 0.60, stress level 0.40) as AI model output. For example, if input such as “I'm motivated this week” text, bright voice, and smiling image is provided, the output is “motivation level 0.90.” The registration unit uses these emotion scores for threshold judgment and rule-based branching to automatically determine the priority of target setting (e.g., prioritize short-term goals, long-term goals, realistic goals). For example, if motivation level is high, short-term goals are prioritized; if anxiety level is high, long-term goals are prioritized, and so on. The registration unit uses the priority determination results for subsequent processing such as initial display of the goal setting screen, generation of advice messages for the user, and automatic adjustment of goal achievement plans. Unlike conventional subjective judgment or simple goal input by humans, the registration unit combines multimodal emotion estimation in high-dimensional feature space and rule-based automatic priority determination to realize individually optimized priority of target setting for each user. For AI model training, supervised learning using emotion-annotated datasets, error backpropagation using loss functions such as cross-entropy loss and mean squared error, and data augmentation (e.g., text paraphrase generation, audio pitch conversion, image rotation) are used to improve model generalization. As a technical effect, the registration unit can grasp the user's emotional state in real time and with high accuracy, automatically optimize the priority of target setting, thereby improving diet continuation rate, goal achievement rate, user experience, and system operational efficiency. Specific application fields include personal diet apps, lifestyle disease prevention programs in medical institutions, corporate health management support services, and mental health care support systems.

The registration unit can preferentially set relevant targets by considering the user's geographic location information when setting the target weight or deadline. The registration unit, for example, preferentially sets relevant targets by considering the user's geographic location information when setting the target weight or deadline. For example, the registration unit sets exercise plans by considering the climate of the region where the user lives. The registration unit can also set goals by considering gyms or exercise facilities that are easily accessible to the user. Furthermore, the registration unit can set meal plans by considering ingredients available in the user's region. Thus, by considering the user's geographic location information, more realistic goal setting becomes possible. Specifically, the registration unit obtains the user's location information data (e.g., GPS coordinates, prefecture/city labels, latitude/longitude vectors), regional climate data (e.g., monthly average temperature, precipitation, snowfall as time-series vectors), surrounding facility data (e.g., list of gyms, exercise facilities, parks within a 2 km radius), and regional ingredient data (e.g., list of ingredients available at local supermarkets, seasonal ingredient labels) from databases or external APIs and inputs them as structured data into the AI model. The registration unit performs preprocessing (e.g., geocoding of location information, time-series aggregation of climate data, categorization of facility data), feature extraction (e.g., exercise facility accessibility score, climate adaptability score, regional ingredient diversity score), and uses machine learning models (e.g., random forest, neural network) and rule-based algorithms to preferentially set target weight, deadline, exercise plan, and meal plan according to the user's geographic location information. Examples of AI input include (1) GPS coordinate vector (e.g., latitude 35.6, longitude 139.7), (2) regional climate vector (e.g., monthly average temperature 20° C., precipitation 100 mm), (3) surrounding facility category array (e.g., “2 gyms,” “1 park”), (4) regional ingredient label array (e.g., “komatsuna,” “mackerel”). Examples of AI output include (1) recommended exercise plan (e.g., “indoor aerobic exercise-focused”), (2) recommended target deadline (e.g., set longer in winter), (3) recommended meal plan (e.g., recipes using local ingredients), (4) facility utilization recommendation label (e.g., “recommend using nearest gym”). For example, in regions with heavy snowfall in winter, the system sets indoor exercise-focused goals, and if there are many gyms nearby, it sets goals recommending gym utilization. Furthermore, the registration unit uses AI output results for subsequent processing such as automatic initial input of the goal setting screen, generation of region-specific advice messages for the user, and automatic adjustment of goal achievement plans. Unlike conventional subjective judgment or simple goal input by humans, the registration unit combines integrated analysis of multidimensional geographic information and regional characteristic data with rule-based automatic priority setting to realize realistic and highly feasible goal setting for each user. For AI model training, classification and regression tasks using geographic information, climate, facility data, and diet achievement data as training data, error backpropagation using loss functions such as cross-entropy loss and mean squared error, and data augmentation (e.g., clustering of location information, adding noise to climate data) are used to improve model generalization. As a technical effect, the registration unit automatically considers the user's geographic constraints and regional characteristics, realizing high-precision, realistic, and highly feasible goal setting, thereby improving goal achievement rate, user satisfaction, and system operational efficiency. Specific application fields include personal diet apps, lifestyle disease prevention programs in medical institutions, corporate health management support services, and region-specific health support systems.

The registration unit can set relevant targets by analyzing the user's social media activity when setting the target weight or deadline. The registration unit, for example, sets relevant targets by analyzing the user's social media activity when setting the target weight or deadline. For example, the registration unit refers to diet goals shared by the user on social media. The registration unit can also refer to diet methods of influencers followed by the user. Furthermore, the registration unit can refer to goals of diet communities the user participates in. Thus, by analyzing the user's social media activity, more relevant goal setting becomes possible. Specifically, the registration unit obtains the user's social media post data (e.g., post text, images, videos, post date/time, hashtags, number of likes/comments for the past year as structured data), follow relationship data (e.g., list of followed influencers, community participation history), and shared goal data (e.g., diet goals declared on SNS, progress report posts) via API and inputs them as structured data into the AI model. The registration unit performs preprocessing (e.g., tokenization of text, feature extraction from images, categorization of hashtags), feature extraction (e.g., frequency of diet-related posts, influencer recommendation method score, community goal distribution), and uses machine learning models (e.g., transformer-based language models, image classification models, clustering algorithms) and rule-based algorithms to recommend target weight, deadline, and diet methods based on the user's social media activity. Examples of AI input include (1) post text vector (e.g., embedding vectors for 100 posts), (2) followed influencer label array (e.g., “strength training,” “low-carb”), (3) community goal label array (e.g., “lose 5 kg in 3 months”). Examples of AI output include (1) recommended target weight (e.g., 55 kg), (2) recommended target deadline (e.g., 90 days), (3) recommended diet method (e.g., “Influencer A's recommended method”), (4) community goal reference label (e.g., “conform to group goal”). For example, if the user has declared “lose 5 kg in 3 months” on SNS, the system automatically sets a similar goal, and if a followed influencer recommends “aerobic exercise+high-protein diet,” that method is prioritized. Furthermore, the registration unit uses AI output results for subsequent processing such as automatic initial input of the goal setting screen, generation of SNS-linked advice messages for the user, and automatic adjustment of goal achievement plans. Unlike conventional subjective judgment or simple goal input by humans, the registration unit combines integrated analysis of multidimensional social media data with rule-based automatic goal setting to realize highly relevant goal setting for each user. For AI model training, classification and regression tasks using SNS post data and diet achievement data as training data, error backpropagation using loss functions such as cross-entropy loss and mean squared error, and data augmentation (e.g., paraphrase generation of post text, image rotation/scaling) are used to improve model generalization. As a technical effect, the registration unit automatically analyzes the user's social media activity and realizes high-precision, highly relevant goal setting, thereby improving goal achievement rate, user satisfaction, and system operational efficiency. Specific application fields include personal diet apps, SNS-linked health support services, corporate health management support services, and community-based diet support systems.

The collection unit can estimate a user's emotion and adjust the timing of data collection based on the estimated emotion. The collection unit, for example, estimates a user's emotion and adjusts the timing of data collection based on the estimated emotion. For example, the collection unit collects data during times when the user is relaxed. The collection unit can also avoid collecting data during times when the user is feeling stressed. Furthermore, the collection unit can collect data after the user has exercised. Thus, by adjusting the timing of data collection according to the user's emotion, more accurate data collection becomes possible. Emotion estimation is realized, for example, by using an emotion engine or generative AI with emotion estimation functionality. Generative AI may be a text generation AI (e.g., LLM) or multimodal generative AI, but is not limited to these examples. Specifically, the collection unit inputs natural language text obtained from the user (e.g., diary comments, meal record comments, questionnaire responses of 50-500 tokens), audio data (e.g., 1-minute audio waveform, 16 kHz sampling), and facial or expression images (e.g., 3×224×224 pixels) into an emotion estimation AI model. The collection unit performs preprocessing on the input data (tokenization of text, spectrogram conversion of audio, facial region extraction from images) and generates feature vectors (e.g., text embedding vector, audio feature vector, image feature vector). The collection unit uses a multimodal emotion estimation model combining transformer-based large language models (e.g., 12 layers, hidden layer 768 dimensions), convolutional neural networks (e.g., ResNet-18), and recurrent neural networks for audio emotion classification (e.g., 2-layer LSTM). The collection unit obtains emotion labels (e.g., “relaxed,” “stress,” “high motivation”), emotion scores (e.g., stress level 0.72, relaxation level 0.85), and emotion change trends (e.g., transition graph for the past 7 days) as AI model output. For example, if input such as “I'm feeling good today” text, calm voice, and smiling image is provided, the output is “relaxation level 0.90,” “stress level 0.10.” In another example, if input such as “I've been busy and tired lately” text, depressed voice, and expressionless image is provided, the output is “stress level 0.80,” “relaxation level 0.20.” The collection unit uses these emotion scores for threshold judgment (e.g., execute data collection if relaxation level is 0.7 or higher, postpone collection if stress level is 0.6 or higher) and rule-based branching to automatically adjust the timing of data collection. Furthermore, the collection unit records the user's emotion change history as a time series and utilizes it as learning data for data collection timing optimization algorithms. Examples of AI input include (1) text embedding vector (e.g., 512 dimensions), (2) audio feature vector (e.g., 128 dimensions), (3) image feature vector (e.g., 2048 dimensions). Examples of AI output include (1) emotion label (e.g., “relaxed”), (2) emotion score (e.g., 0.85), (3) recommended collection timing (e.g., “20:00-21:00”). The collection unit automatically controls the data collection scheduler based on AI output results, minimizing the user's psychological burden while maximizing data comprehensiveness and accuracy. Unlike conventional fixed schedules or manual adjustment of collection timing, the collection unit combines multimodal emotion estimation in high-dimensional feature space and rule-based automatic timing optimization to realize individually optimized data collection timing for each user. For AI model training, supervised learning using large-scale emotion-annotated datasets, error backpropagation using loss functions such as cross-entropy loss and mean squared error, and data augmentation (e.g., text paraphrase generation, audio pitch conversion, image rotation) are used to improve model generalization and robustness. As a technical effect, the collection unit can grasp the user's emotional state in real time and with high accuracy, greatly improving the realism and accuracy of data collection timing, thereby improving data quality, reducing user burden, and improving overall system operational efficiency. Specific application fields include personal health management apps, lifestyle disease prevention programs in medical institutions, corporate health management support services, and mental health care support systems.

The collection unit can analyze the user's past data collection history and select an optimal collection method. The collection unit, for example, analyzes the user's past data collection history and selects an optimal collection method. For example, the collection unit refers to data collection methods that the user has succeeded with in the past. The collection unit can also avoid data collection methods that the user has failed with in the past. Furthermore, the collection unit can propose optimal timing based on the user's past data collection history. Thus, by analyzing past data collection history, the optimal data collection method can be selected. Specifically, the collection unit obtains data collection history recorded as a time series for each user (e.g., collection date/time of meal images for the past year, frequency of exercise data acquisition, type of collection device, collection success rate, error codes for failed collections, user's subjective satisfaction score as structured data) from the database. The collection unit performs feature extraction (e.g., collection success rate, average collection interval, collection efficiency by device, labeling of failure factors) on these history data and uses machine learning models (e.g., decision trees, random forests, clustering algorithms) and rule-based algorithms to automatically analyze the user's past success and failure patterns. Examples of AI input include (1) time-series vector of collection date/time (e.g., 365 days), (2) category label array of collection devices (e.g., “smartphone,” “wearable”), (3) collection success/failure flag array (e.g., 1=success, 0=failure), (4) collection satisfaction score (e.g., 0.8, 0.6, 0.9). Examples of AI output include (1) recommended collection method label (e.g., “wearable prioritized,” “image +audio combined”), (2) recommended collection timing (e.g., “before breakfast,” “immediately after exercise”), (3) failure risk score (e.g., 0.15), (4) recommended collection frequency (e.g., 3 times per day). For example, if “smartphone image collection before breakfast” has a high success rate in the past, the same method is recommended, and if “wearable cooperation at night” has many failures, that method is excluded. Furthermore, the collection unit uses AI output results for subsequent processing such as automatic adjustment of the collection scheduler, generation of collection method advice messages for the user, and automatic optimization of collection frequency. Unlike conventional subjective history reference or simple fixed schedules by humans, the collection unit autonomously performs feature extraction and pattern recognition of high-dimensional history data by AI, realizing automatic proposal of individually optimized data collection methods for each user. For AI model training, classification and regression tasks using past collection history and collection success/failure as training data, error backpropagation using loss functions such as cross-entropy loss and mean squared error, and data augmentation (e.g., time-series shift of history data, sampling of device labels) are used to improve model generalization. As a technical effect, the collection unit analyzes the user's past behavior and achievement data with high accuracy and automates individually optimized and realistic data collection methods, thereby improving collection success rate, homogenizing data quality, reducing user burden, and improving system operational efficiency. Specific application fields include personal health management apps, lifestyle disease prevention programs in medical institutions, corporate health management support services, and personal training support systems.

The collection unit can perform filtering based on the user's current living conditions or areas of interest during data collection. The collection unit, for example, performs filtering based on the user's current living conditions or areas of interest during data collection. For example, the collection unit collects data by considering the user's current meal content. The collection unit can also collect data by considering the user's current exercise status. Furthermore, the collection unit can collect data by considering the user's areas of interest (e.g., specific diet methods). Thus, by collecting data based on the user's current living conditions or areas of interest, more relevant data can be collected. Specifically, the collection unit obtains recent meal records from the user (e.g., meal images, ingredient labels, calorie intake for the past 7 days), exercise records (e.g., exercise type, intensity, time, calories burned for one week), lifestyle habit data (e.g., sleep time, wake/sleep time, drinking/smoking habits), and areas of interest selected by the user (e.g., category labels such as “low-carb,” “strength training,” “fasting”) from the database and inputs them as structured data into the AI model. The collection unit performs preprocessing (e.g., missing value imputation, category conversion, normalization), feature extraction (e.g., meal pattern vector, exercise frequency score, one-hot vector of areas of interest), and uses machine learning models (e.g., random forest, neural network, rule-based filter) to prioritize and filter data collection targets according to the user's current living conditions and areas of interest. Examples of AI input include (1) meal content vector (e.g., labels of staple food, main dish, side dish for 7 days), (2) exercise status vector (e.g., aerobic exercise 3 times a week, strength training 2 times a week), (3) category label of areas of interest (e.g., “low-carb”). Examples of AI output include (1) recommended data type for collection (e.g., “meal images prioritized,” “exercise data prioritized”), (2) recommended collection frequency (e.g., 3 times per day), (3) unnecessary data exclusion flag (e.g., exclude snack data), (4) area-of-interest-specific collection label (e.g., “strength training record enhancement”). For example, if the user is interested in “low-carb,” detailed collection of carbohydrate intake is prioritized, and if the user has an active exercise habit, high-frequency collection of exercise data is recommended. Furthermore, the collection unit uses AI output results for subsequent processing such as automatic adjustment of input items on the collection screen, generation of collection advice messages for the user, and automatic optimization of collection frequency. Unlike conventional uniform data collection or manual selection of collection items by humans, the collection unit combines integrated analysis of multidimensional living conditions and areas of interest data with rule-based automatic filtering to realize highly relevant data collection for each user. For AI model training, classification and regression tasks using living conditions, areas of interest data, and collection achievement data as training data, error backpropagation using loss functions such as cross-entropy loss and mean squared error, and data augmentation (e.g., sampling of meal patterns, randomization of area-of-interest labels) are used to improve model generalization. As a technical effect, the collection unit automatically considers the user's current living conditions and areas of interest, realizing high-precision, highly relevant data collection, thereby improving data quality, reducing user burden, and improving system operational efficiency. Specific application fields include personal health management apps, lifestyle disease prevention programs in medical institutions, corporate health management support services, and personal training support systems.

The collection unit can estimate a user's emotion and determine the priority of data to be collected based on the estimated emotion. The collection unit, for example, estimates a user's emotion and determines the priority of data to be collected based on the estimated emotion. For example, if the user is highly motivated, the collection unit prioritizes collecting exercise data. If the user is feeling stressed, the collection unit can prioritize collecting meal data. Furthermore, if the user is relaxed, the collection unit can collect all data in a balanced manner. Thus, by determining the priority of data to be collected according to the user's emotion, more effective data collection becomes possible. Emotion estimation is realized, for example, by using an emotion engine or generative AI with emotion estimation functionality. Generative AI may be a text generation AI (e.g., LLM) or multimodal generative AI, but is not limited to these examples. Specifically, the collection unit inputs natural language text obtained from the user (e.g., diary comments, meal record comments, questionnaire responses of 50-500 tokens), audio data (e.g., 1-minute audio waveform, 16 kHz sampling), and facial or expression images (e.g., 3×224×224 pixels) into an emotion estimation AI model. The collection unit performs preprocessing on the input data (tokenization of text, spectrogram conversion of audio, facial region extraction from images) and generates feature vectors (e.g., text embedding vector, audio feature vector, image feature vector). The collection unit uses a multimodal emotion estimation model combining transformer-based large language models (e.g., 12 layers, hidden layer 768 dimensions), convolutional neural networks (e.g., ResNet-18), and recurrent neural networks for audio emotion classification (e.g., 2-layer LSTM). The collection unit obtains emotion labels (e.g., “high motivation,” “stress,” “relaxed”), emotion scores (e.g., motivation level 0.85, stress level 0.60, relaxation level 0.40) as AI model output. For example, if input such as “I'm motivated this week” text, bright voice, and smiling image is provided, the output is “motivation level 0.90.” The collection unit uses these emotion scores for threshold judgment and rule-based branching to automatically determine the priority of data to be collected (e.g., prioritize exercise data, prioritize meal data, balanced collection). For example, if motivation level is high, exercise data is prioritized; if stress level is high, meal data is prioritized, and so on. The collection unit uses the priority determination results for subsequent processing such as initial display of the collection screen, generation of advice messages for the user, and automatic adjustment of collection plans. Unlike conventional subjective judgment or simple selection of collection items by humans, the collection unit combines multimodal emotion estimation in high-dimensional feature space and rule-based automatic priority determination to realize individually optimized priority of data collection for each user. For AI model training, supervised learning using emotion-annotated datasets, error backpropagation using loss functions such as cross-entropy loss and mean squared error, and data augmentation (e.g., text paraphrase generation, audio pitch conversion, image rotation) are used to improve model generalization. As a technical effect, the collection unit can grasp the user's emotional state in real time and with high accuracy, automatically optimize the priority of data collection, thereby improving data quality, user experience, and system operational efficiency. Specific application fields include personal health management apps, lifestyle disease prevention programs in medical institutions, corporate health management support services, and mental health care support systems.

The collection unit can preferentially collect relevant data by considering the user's geographic location information during data collection. The collection unit, for example, preferentially collects relevant data by considering the user's geographic location information during data collection. For example, the collection unit collects meal data by considering ingredients available in the user's region. The collection unit can also collect exercise data by considering exercise facilities that are easily accessible to the user. Furthermore, the collection unit can collect exercise data by considering the climate of the region where the user lives. Thus, by considering the user's geographic location information, more relevant data collection becomes possible. Specifically, the collection unit obtains the user's location information data (e.g., GPS coordinates, prefecture/city labels, latitude/longitude vectors), regional climate data (e.g., monthly average temperature, precipitation, snowfall as time-series vectors), surrounding facility data (e.g., list of gyms, exercise facilities, parks within a 2 km radius), and regional ingredient data (e.g., list of ingredients available at local supermarkets, seasonal ingredient labels) from databases or external APIs and inputs them as structured data into the AI model. The collection unit performs preprocessing (e.g., geocoding of location information, time-series aggregation of climate data, categorization of facility data), feature extraction (e.g., exercise facility accessibility score, climate adaptability score, regional ingredient diversity score), and uses machine learning models (e.g., random forest, neural network) and rule-based algorithms to preferentially set data collection targets according to the user's geographic location information. Examples of AI input include (1) GPS coordinate vector (e.g., latitude 35.6, longitude 139.7), (2) regional climate vector (e.g., monthly average temperature 20° C., precipitation 100 mm), (3) surrounding facility category array (e.g., “2 gyms,” “1 park”), (4) regional ingredient label array (e.g., “komatsuna,” “mackerel”). Examples of AI output include (1) recommended data type for collection (e.g., “meal images focused on regional ingredients,” “indoor exercise data”), (2) recommended collection frequency (e.g., once a week in winter, three times a week in summer), (3) facility utilization recommendation label (e.g., “prioritize data from nearest gym”). For example, in regions with heavy snowfall in winter, the system prioritizes collection of indoor exercise data, and if there are many gyms nearby, it prioritizes collection of data from gym utilization. Furthermore, the collection unit uses AI output results for subsequent processing such as automatic initial input of the collection screen, generation of region-specific advice messages for the user, and automatic adjustment of collection plans. Unlike conventional subjective judgment or simple selection of collection items by humans, the collection unit combines integrated analysis of multidimensional geographic information and regional characteristic data with rule-based automatic priority setting to realize realistic and highly feasible data collection for each user. For AI model training, classification and regression tasks using geographic information, climate, facility data, and collection achievement data as training data, error backpropagation using loss functions such as cross-entropy loss and mean squared error, and data augmentation (e.g., clustering of location information, adding noise to climate data) are used to improve model generalization. As a technical effect, the collection unit automatically considers the user's geographic constraints and regional characteristics, realizing high-precision, realistic, and highly feasible data collection, thereby improving data quality, user satisfaction, and system operational efficiency. Specific application fields include personal health management apps, lifestyle disease prevention programs in medical institutions, corporate health management support services, and region-specific health support systems.

The collection unit can collect relevant data by analyzing the user's social media activity during data collection. The collection unit, for example, collects relevant data by analyzing the user's social media activity during data collection. For example, the collection unit refers to meal content shared by the user on social media. The collection unit can also refer to exercise methods of influencers followed by the user. Furthermore, the collection unit can refer to data from diet communities the user participates in. Thus, by analyzing the user's social media activity, more relevant data collection becomes possible. Specifically, the collection unit obtains the user's social media post data (e.g., post text, images, videos, post date/time, hashtags, number of likes/comments for the past year as structured data), follow relationship data (e.g., list of followed influencers, community participation history), and shared goal data (e.g., diet goals declared on SNS, progress report posts) via API and inputs them as structured data into the AI model. The collection unit performs preprocessing (e.g., tokenization of text, feature extraction from images, categorization of hashtags), feature extraction (e.g., frequency of diet-related posts, influencer recommendation method score, community goal distribution), and uses machine learning models (e.g., transformer-based language models, image classification models, clustering algorithms) and rule-based algorithms to recommend data collection targets based on the user's social media activity. Examples of AI input include (1) post text vector (e.g., embedding vectors for 100 posts), (2) followed influencer label array (e.g., “strength training,” “low-carb”), (3) community goal label array (e.g., “lose 5 kg in 3 months”). Examples of AI output include (1) recommended data type for collection (e.g., “SNS-shared meal images,” “influencer-recommended exercise data”), (2) recommended collection frequency (e.g., high frequency during community events), (3) community goal reference label (e.g., “conform to group goal”). For example, if the user has declared “lose 5 kg in 3 months” on SNS, the system automatically sets similar data collection, and if a followed influencer recommends “aerobic exercise+high-protein diet,” that data collection is prioritized. Furthermore, the collection unit uses AI output results for subsequent processing such as automatic initial input of the collection screen, generation of SNS-linked advice messages for the user, and automatic adjustment of collection plans. Unlike conventional subjective judgment or simple selection of collection items by humans, the collection unit combines integrated analysis of multidimensional social media data with rule-based automatic data collection setting to realize highly relevant data collection for each user. For AI model training, classification and regression tasks using SNS post data and collection achievement data as training data, error backpropagation using loss functions such as cross-entropy loss and mean squared error, and data augmentation (e.g., paraphrase generation of post text, image rotation/scaling) are used to improve model generalization. As a technical effect, the collection unit automatically analyzes the user's social media activity and realizes high-precision, highly relevant data collection, thereby improving data quality, user satisfaction, and system operational efficiency. Specific application fields include personal health management apps, SNS-linked health support services, corporate health management support services, and community-based diet support systems.

The analysis unit can estimate a user's emotion and adjust the data analysis method based on the estimated emotion. For example, the analysis unit estimates the user's emotion and adjusts the data analysis method according to the estimated emotion. For instance, when the user is relaxed, the analysis unit performs detailed data analysis. When the user is stressed, the analysis unit can perform concise data analysis. Furthermore, when the user is highly motivated, the analysis unit can perform rigorous data analysis. By adjusting the data analysis method according to the user's emotion, more accurate data analysis becomes possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the analysis unit inputs natural language text obtained from the user (e.g., diary comments, meal record comments, survey responses of 50 to 500 tokens), audio data (e.g., 1-minute audio waveform, 16 kHz sampling), and facial or expression images (e.g., 3×224×224 pixels) into an emotion estimation AI model. The analysis unit performs preprocessing on the input data (tokenization of text, spectrogram conversion of audio, extraction of facial regions from images) and generates feature vectors (e.g., text embedding vectors, audio feature vectors, image feature vectors). The analysis unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The analysis unit obtains outputs from the AI model such as emotion labels (e.g., “relaxed”, “stressed”, “high motivation”), emotion scores (e.g., stress level 0.72, relaxation level 0.85), and emotion change trends (e.g., transition graph over the past 7 days). For example, when text such as “I feel good today”, a calm voice, and a smiling image are input, the output may be “relaxation level 0.90”, “stress level 0.10”. In another example, when text such as “I've been busy and tired lately”, a depressed voice, and an expressionless image are input, the output may be “stress level 0.80”, “relaxation level 0.20”. The analysis unit uses these emotion scores for threshold judgment (e.g., detailed analysis if relaxation level is 0.7 or higher, simple analysis if stress level is 0.6 or higher, rigorous analysis if motivation level is 0.8 or higher) and rule-based branching processing, and automatically switches the data analysis method (e.g., number of layers in the analysis model, selection of features, analysis frequency, analysis granularity). For example, detailed analysis uses multilayer neural network analysis with all features, simple analysis uses decision tree analysis with only major features, and rigorous analysis applies ensemble learning or Bayesian optimization. The analysis unit sends the analysis results to the suggestion unit and modification unit for use in generating individually optimized meal and exercise plans and progress management. Unlike conventional uniform analysis processing or manual selection of analysis methods, the analysis unit realizes user-optimized data analysis by combining multimodal emotion estimation in high-dimensional feature space and rule-based automatic optimization of analysis methods. For AI model training, supervised learning using large-scale emotion-annotated datasets, error backpropagation using cross-entropy loss or mean squared error as the loss function, and data augmentation (e.g., text paraphrase generation, audio pitch conversion, image rotation) are utilized to enhance model generalization and robustness. As a technical effect, the analysis unit can grasp the user's emotional state in real time and with high accuracy, greatly improving the realism, accuracy, and efficiency of analysis methods, thereby achieving improved data analysis accuracy, reduced user burden, and enhanced operational efficiency of the entire system. Specific application fields include personal health management apps, lifestyle disease prevention programs at medical institutions, corporate health management support services, and mental health care support systems.

The analysis unit can optimize the analysis algorithm by referring to past analysis data during data analysis. For example, the analysis unit optimizes the analysis algorithm by referring to past analysis data during data analysis. For instance, the analysis unit optimizes the analysis algorithm by referring to past successful cases. The analysis unit can also optimize the analysis algorithm to avoid past failure cases. Furthermore, the analysis unit can propose optimal analysis methods based on past data analysis history. By referring to past analysis data, optimization of the analysis algorithm becomes possible. Specifically, the analysis unit acquires past analysis history data recorded in chronological order for each user (e.g., application history of analysis models to weight, exercise, and meal data over the past year, input feature vectors for each analysis, parameters of used models, accuracy indicators of analysis results, user feedback scores, error codes in case of failure, etc.) from a database. The analysis unit performs feature extraction on these history data (e.g., accuracy trends for each model, labeling of failure factors, analysis success rate, time-series changes in user satisfaction scores), and uses machine learning models (e.g., decision trees, random forests, Bayesian optimization algorithms, meta-learning models) or rule-based algorithms to automatically analyze past success and failure patterns. Examples of AI inputs include (1) time-series vectors of analysis history (e.g., model ID, accuracy, failure flag for 365 days), (2) input feature vectors (e.g., integrated vectors of meal, exercise, and weight data), (3) user feedback scores (e.g., 0.8, 0.6, 0.9), and (4) error code arrays (e.g., 0=success, 1=data missing, 2=model mismatch). Examples of AI outputs include (1) recommended analysis algorithm labels (e.g., “LSTM+CNN combination”, “decision tree”, “ensemble learning”), (2) recommended hyperparameters (e.g., learning rate 0.01, number of layers 4), (3) failure risk score (e.g., 0.12), and (4) recommended analysis frequency (e.g., once per day). For example, if high-accuracy analysis results were obtained in the past using the “LSTM+CNN combination model”, the same model is recommended, and if there were many failures with “decision tree”, that method is excluded. Furthermore, based on the AI output results, the analysis unit performs subsequent processing such as automatic switching of analysis algorithms, parameter optimization, automatic adjustment of analysis frequency, and generation of explanatory messages for analysis methods to users. Unlike conventional subjective model selection by humans or simple application of fixed algorithms, the analysis unit autonomously extracts features and recognizes patterns from high-dimensional history data using AI, thereby realizing automatic selection and optimization of analysis algorithms optimized for each user. For AI model training, classification and regression tasks using past analysis history and analysis accuracy/failure as training data, error backpropagation using cross-entropy loss or mean squared error as the loss function, and data augmentation (e.g., time-series shifting of history data, sampling of model parameters) are utilized to enhance model generalization and robustness. As a technical effect, the analysis unit can analyze the user's past analysis history with high accuracy and realize automatic selection and optimization of individually optimized analysis algorithms, thereby achieving improved analysis accuracy, reduced failure rate, improved user satisfaction, and enhanced system operational efficiency. Specific application fields include personal health management apps, lifestyle disease prevention programs at medical institutions, corporate health management support services, and personal training support systems.

The analysis unit can improve the accuracy of analysis based on the user's health status and lifestyle habits during data analysis. For example, the analysis unit improves the accuracy of analysis based on the user's health status and lifestyle habits during data analysis. For instance, the analysis unit performs data analysis by referring to the user's health checkup results. The analysis unit can also perform data analysis by considering the user's lifestyle habits (diet, exercise, sleep). Furthermore, the analysis unit can perform data analysis by considering the user's medical history. By improving the accuracy of analysis based on the user's health status and lifestyle habits, more accurate data analysis becomes possible. Specifically, the analysis unit acquires health checkup data obtained from the user (e.g., height, weight, BMI, blood pressure, blood glucose, cholesterol, liver function values as numerical vectors), lifestyle habit data (e.g., one week of meal records, exercise frequency, average sleep time, presence or absence of drinking/smoking habits), and medical history data (e.g., past disease labels, treatment history, medication information) from a database and inputs them as structured data into the AI model. The analysis unit performs preprocessing on these data (e.g., missing value imputation, normalization, category conversion), feature extraction (e.g., health risk score, lifestyle habit score, disease risk label), and uses machine learning models (e.g., random forest, logistic regression, neural network) or rule-based algorithms to select analysis methods and optimize analysis parameters according to the user's health status and lifestyle habits. Examples of AI inputs include (1) health checkup numerical vectors (e.g., 10 dimensions), (2) lifestyle habit category label arrays (e.g., “skipping breakfast”, “exercise twice a week”, “6 hours sleep”), and (3) medical history label arrays (e.g., “hypertension”, “diabetes”). Examples of AI outputs include (1) recommended analysis model labels (e.g., “multilayer perceptron”, “random forest”), (2) recommended analysis parameters (e.g., feature selection list, analysis frequency), (3) risk warning labels (e.g., “high risk of hypertension”), and (4) analysis accuracy score (e.g., 0.92). For example, if hypertension is found in a health checkup, an analysis model that emphasizes features sensitive to blood pressure fluctuations is automatically selected, and if there is a tendency for sleep deprivation, analysis that emphasizes sleep-related features is performed. Furthermore, based on the AI output results, the analysis unit performs subsequent processing such as automatic switching of analysis methods, optimization of analysis parameters, display of health risk warnings to users, and adjustment of the detail level of analysis results. Unlike conventional subjective selection of analysis methods by humans or simple uniform analysis, the analysis unit realizes highly accurate and safe data analysis for each user by combining integrated analysis of multidimensional health and lifestyle habit data with rule-based automatic optimization. For AI model training, classification and regression tasks using health checkup data and analysis outcome data as training data, error backpropagation using cross-entropy loss or mean squared error as the loss function, and data augmentation (e.g., adding noise to health checkup values, sampling lifestyle habit patterns) are utilized to enhance model generalization. As a technical effect, the analysis unit automatically avoids health risks for users and realizes highly accurate, realistic, and safe analysis, thereby achieving reduced risk of health damage, improved analysis accuracy, improved user satisfaction, and enhanced system operational efficiency. Specific application fields include personal health management apps, lifestyle disease prevention programs at medical institutions, corporate health management support services, and health promotion programs by insurance companies.

The analysis unit can estimate a user's emotion and adjust the display method of analysis results based on the estimated emotion. For example, the analysis unit estimates the user's emotion and adjusts the display method of analysis results according to the estimated emotion. For instance, when the user is nervous, the analysis unit provides a simple and highly visible display method. When the user is relaxed, the analysis unit can provide a display method that includes detailed information. Furthermore, when the user is in a hurry, the analysis unit can provide a display method that emphasizes key points. By adjusting the display method of analysis results according to the user's emotion, more appropriate information provision becomes possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the analysis unit inputs natural language text obtained from the user (e.g., diary comments, meal record comments, survey responses of 50 to 500 tokens), audio data (e.g., 1-minute audio waveform, 16 kHz sampling), and facial or expression images (e.g., 3×224×224 pixels) into an emotion estimation AI model. The analysis unit performs preprocessing on the input data (tokenization of text, spectrogram conversion of audio, extraction of facial regions from images) and generates feature vectors (e.g., text embedding vectors, audio feature vectors, image feature vectors). The analysis unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The analysis unit obtains outputs from the AI model such as emotion labels (e.g., “nervous”, “relaxed”, “in a hurry”), emotion scores (e.g., nervousness level 0.80, relaxation level 0.60, hurry level 0.70), and emotion change trends (e.g., transition graph over the past 7 days). For example, when text such as “I'm nervous today”, a fast-spoken voice, and a tense facial image are input, the output may be “nervousness level 0.85”. The analysis unit uses these emotion scores for threshold judgment and rule-based branching processing, and automatically determines the display method of analysis results (e.g., simple display, detailed display, key point display). For example, when nervousness level is high, a simple display with minimal graphs and numbers is selected; when relaxation level is high, a detailed display with analysis graphs and explanatory text is added; when hurry level is high, a display emphasizing only key points is selected. The analysis unit reflects the display method decision results in the user interface and uses them for subsequent processing such as generating advice messages to users and automatic summarization of analysis results. Unlike conventional uniform information display or manual selection of display methods, the analysis unit realizes user-optimized analysis result display by combining multimodal emotion estimation in high-dimensional feature space and rule-based automatic display optimization. For AI model training, supervised learning using emotion-annotated datasets, error backpropagation using cross-entropy loss or mean squared error as the loss function, and data augmentation (e.g., text paraphrase generation, audio pitch conversion, image rotation) are utilized to enhance model generalization. As a technical effect, the analysis unit can grasp the user's emotional state in real time and with high accuracy, greatly improving the realism and appropriateness of analysis result display, thereby achieving improved user experience, improved information transmission efficiency, and enhanced system operational efficiency. Specific application fields include personal health management apps, lifestyle disease prevention programs at medical institutions, corporate health management support services, and mental health care support systems.

The analysis unit can improve the accuracy of analysis by considering the user's geographic location information during data analysis. For example, the analysis unit improves the accuracy of analysis by considering the user's geographic location information during data analysis. For instance, the analysis unit performs data analysis by considering the climate of the region where the user lives. The analysis unit can also perform data analysis by considering exercise facilities that are easily accessible to the user. Furthermore, the analysis unit can perform data analysis by considering local ingredients available in the user's region. By considering the user's geographic location information, more accurate data analysis becomes possible. Specifically, the analysis unit acquires the user's location information data (e.g., GPS coordinates, prefecture/city labels, latitude/longitude vectors), regional climate data (e.g., monthly average temperature, precipitation, snowfall as time-series vectors), surrounding facility data (e.g., list of gyms, exercise facilities, parks within a 2 km radius), and local ingredient data (e.g., list of ingredients handled by local supermarkets, seasonal ingredient labels) from databases or external APIs and inputs them as structured data into the AI model. The analysis unit performs preprocessing on these data (e.g., geocoding of location information, time-series aggregation of climate data, categorization of facility data), feature extraction (e.g., exercise facility accessibility score, climate adaptability score, local ingredient diversity score), and uses machine learning models (e.g., random forest, neural network) or rule-based algorithms to select analysis methods and optimize analysis parameters according to the user's geographic location information. Examples of AI inputs include (1) GPS coordinate vectors (e.g., latitude 35.6, longitude 139.7), (2) regional climate vectors (e.g., monthly average temperature 20° C., precipitation 100 mm), (3) surrounding facility category arrays (e.g., “2 gyms”, “1 park”), and (4) local ingredient label arrays (e.g., “komatsuna”, “mackerel”). Examples of AI outputs include (1) recommended analysis model labels (e.g., “climate-adaptive neural network”), (2) recommended analysis parameters (e.g., weighting of climate features), (3) facility usage recommendation labels (e.g., “emphasize nearest gym data”), and (4) analysis accuracy score (e.g., 0.93). For example, in regions with heavy snowfall in winter, analysis emphasizing indoor exercise data is performed, and in areas with many nearby gyms, analysis emphasizing gym usage data is performed. Furthermore, based on the AI output results, the analysis unit performs subsequent processing such as automatic switching of analysis methods, optimization of analysis parameters, generation of region-specific advice messages to users, and reflection of regional characteristics in analysis results. Unlike conventional uniform analysis processing or manual consideration of regional characteristics, the analysis unit realizes realistic and highly accurate data analysis for each user by combining integrated analysis of multidimensional geographic and regional characteristic data with rule-based automatic optimization. For AI model training, classification and regression tasks using geographic information, climate, facility data, and analysis outcome data as training data, error backpropagation using cross-entropy loss or mean squared error as the loss function, and data augmentation (e.g., clustering of location information, adding noise to climate data) are utilized to enhance model generalization. As a technical effect, the analysis unit automatically considers the user's geographic constraints and regional characteristics, and realizes highly accurate, realistic, and feasible data analysis, thereby achieving improved analysis accuracy, improved user satisfaction, and enhanced system operational efficiency. Specific application fields include personal health management apps, lifestyle disease prevention programs at medical institutions, corporate health management support services, and region-specific health support systems.

The analysis unit can analyze the user's social media activity and analyze relevant data during data analysis. For example, the analysis unit analyzes the user's social media activity and analyzes relevant data during data analysis. For instance, the analysis unit refers to meal content shared by the user on social media. The analysis unit can also refer to exercise methods of influencers followed by the user. Furthermore, the analysis unit can refer to data from diet communities in which the user participates. By analyzing the user's social media activity, more relevant data analysis becomes possible. Specifically, the analysis unit acquires the user's social media post data (e.g., post texts, images, videos, post dates, hashtags, number of likes/comments over the past year as structured data), follow relationship data (e.g., list of followed influencers, community participation history), and shared goal data (e.g., diet goals declared on SNS, progress report posts) via API and inputs them as structured data into the AI model. The analysis unit performs preprocessing on these data (e.g., tokenization of text, feature extraction from images, categorization of hashtags), feature extraction (e.g., frequency of diet-related posts, influencer recommendation method score, community goal distribution), and uses machine learning models (e.g., transformer-based language models, image classification models, clustering algorithms) or rule-based algorithms to select analysis target data and optimize analysis methods based on the user's social media activity. Examples of AI inputs include (1) post text vectors (e.g., embedding vectors for 100 posts), (2) followed influencer label arrays (e.g., “muscle training”, “low-carb”), and (3) community goal label arrays (e.g., “lose 5 kg in 3 months”). Examples of AI outputs include (1) recommended analysis data types (e.g., “SNS shared meal images”, “influencer recommended exercise data”), (2) recommended analysis frequency (e.g., high frequency during community events), (3) community goal reference labels (e.g., “conform to group goal”), and (4) analysis accuracy score (e.g., 0.91). For example, if the user declares “lose 5 kg in 3 months” on SNS, the same data analysis is automatically set, and if a followed influencer recommends “aerobic exercise+high-protein diet”, that data analysis is prioritized. Furthermore, based on the AI output results, the analysis unit performs subsequent processing such as automatic input of initial values on the analysis screen, generation of SNS cooperation advice messages to users, and automatic adjustment of analysis plans. Unlike conventional subjective judgment by humans or simple selection of analysis items, the analysis unit realizes highly relevant data analysis for each user by combining integrated analysis of multidimensional social media data with rule-based automatic analysis setting. For AI model training, classification and regression tasks using SNS post data and analysis outcome data as training data, error backpropagation using cross-entropy loss or mean squared error as the loss function, and data augmentation (e.g., paraphrase generation of post texts, image rotation/scaling) are utilized to enhance model generalization. As a technical effect, the analysis unit automatically analyzes the user's social media activity and realizes highly accurate, relevant data analysis, thereby achieving improved analysis accuracy, improved user satisfaction, and enhanced system operational efficiency. Specific application fields include personal health management apps, SNS-linked health support services, corporate health management support services, and community-based diet support systems.

The suggestion unit can estimate a user's emotion and adjust the expression method of suggestions based on the estimated emotion. For example, the suggestion unit estimates the user's emotion and adjusts the expression method of suggestions according to the estimated emotion. For instance, when the user is relaxed, the suggestion unit provides detailed suggestions. When the user is stressed, the suggestion unit can provide concise suggestions. Furthermore, when the user is highly motivated, the suggestion unit can provide rigorous suggestions. By adjusting the expression method of suggestions according to the user's emotion, more appropriate suggestions become possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the suggestion unit inputs natural language text obtained from the user (e.g., comments when entering diet goals, diaries, survey responses of 50 to 500 tokens), audio data (e.g., 1-minute audio waveform, 16 kHz sampling), and facial or expression images (e.g., 3×224×224 pixels) into an emotion estimation AI model. The suggestion unit performs preprocessing on the input data (tokenization of text, spectrogram conversion of audio, extraction of facial regions from images) and generates feature vectors (e.g., text embedding vectors, audio feature vectors, image feature vectors). The suggestion unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The suggestion unit obtains outputs from the AI model such as emotion labels (e.g., “relaxed”, “stressed”, “high motivation”), emotion scores (e.g., stress level 0.72, relaxation level 0.85), and emotion change trends (e.g., transition graph over the past 7 days). For example, when text such as “I feel good today”, a calm voice, and a smiling image are input, the output may be “relaxation level 0.90”, “stress level 0.10”. In another example, when text such as “I've been busy and tired lately”, a depressed voice, and an expressionless image are input, the output may be “stress level 0.80”, “relaxation level 0.20”. The suggestion unit uses these emotion scores for threshold judgment (e.g., detailed suggestion if relaxation level is 0.7 or higher, concise suggestion if stress level is 0.6 or higher, rigorous suggestion if motivation level is 0.8 or higher) and rule-based branching processing, and automatically switches the expression method of suggestions (e.g., suggestions with detailed explanations, concise suggestions with only key points, rigorous suggestions emphasizing numerical evidence). The suggestion unit reflects the suggestion content in the user interface and uses it for subsequent processing such as generating advice messages to users and automatic summarization of suggestion content. Unlike conventional uniform suggestion display or manual selection of expression methods, the suggestion unit realizes user-optimized suggestion expression by combining multimodal emotion estimation in high-dimensional feature space and rule-based automatic expression optimization. For AI model training, supervised learning using emotion-annotated datasets, error backpropagation using cross-entropy loss or mean squared error as the loss function, and data augmentation (e.g., text paraphrase generation, audio pitch conversion, image rotation) are utilized to enhance model generalization. As a technical effect, the suggestion unit can grasp the user's emotional state in real time and with high accuracy, greatly improving the realism and appropriateness of suggestion expression, thereby achieving improved user experience, improved information transmission efficiency, and enhanced system operational efficiency. Specific application fields include personal health management apps, lifestyle disease prevention programs at medical institutions, corporate health management support services, and mental health care support systems.

The suggestion unit can customize the content of suggestions based on the user's health status and lifestyle habits at the time of suggestion. For example, the suggestion unit customizes the content of suggestions based on the user's health status and lifestyle habits at the time of suggestion. For instance, the suggestion unit customizes the content of suggestions by referring to the user's health checkup results. The suggestion unit can also customize the content of suggestions by considering the user's lifestyle habits (diet, exercise, sleep). Furthermore, the suggestion unit can customize the content of suggestions by considering the user's medical history. By customizing the content of suggestions based on the user's health status and lifestyle habits, more effective suggestions become possible. Specifically, the suggestion unit acquires health checkup data obtained from the user (e.g., height, weight, BMI, blood pressure, blood glucose, cholesterol, liver function values as numerical vectors), lifestyle habit data (e.g., one week of meal records, exercise frequency, average sleep time, presence or absence of drinking/smoking habits), and medical history data (e.g., past disease labels, treatment history, medication information) from a database and inputs them as structured data into the AI model. The suggestion unit performs preprocessing on these data (e.g., missing value imputation, normalization, category conversion), feature extraction (e.g., health risk score, lifestyle habit score, disease risk label), and uses machine learning models (e.g., random forest, logistic regression, neural network) or rule-based algorithms to customize the content of suggestions according to the user's health status and lifestyle habits. Examples of AI inputs include (1) health checkup numerical vectors (e.g., 10 dimensions), (2) lifestyle habit category label arrays (e.g., “skipping breakfast”, “exercise twice a week”, “6 hours sleep”), and (3) medical history label arrays (e.g., “hypertension”, “diabetes”). Examples of AI outputs include (1) recommended meal labels (e.g., “low-salt Japanese cuisine”), (2) recommended exercise plans (e.g., “light aerobic exercise”), (3) risk warning labels (e.g., “rapid weight loss not allowed”), and (4) recommended lifestyle improvement actions (e.g., “extend sleep time”). For example, if hypertension is found in a health checkup, suggestions for low-salt meals and light exercise are recommended, and if there is a tendency for sleep deprivation, suggestions prioritizing sleep improvement are made. Furthermore, based on the AI output results, the suggestion unit performs subsequent processing such as input restrictions on the suggestion screen (e.g., automatic blocking of dangerous suggestions), display of health risk warnings to users, and automatic adjustment of suggestion content. Unlike conventional subjective advice by humans or simple suggestions, the suggestion unit realizes safe and feasible suggestions for each user by combining integrated analysis of multidimensional health and lifestyle habit data with rule-based automatic customization. For AI model training, classification and regression tasks using health checkup data and suggestion outcome data as training data, error backpropagation using cross-entropy loss or mean squared error as the loss function, and data augmentation (e.g., adding noise to health checkup values, sampling lifestyle habit patterns) are utilized to enhance model generalization. As a technical effect, the suggestion unit automatically avoids health risks for users and realizes highly accurate, realistic, and safe suggestions, thereby achieving reduced risk of health damage, improved suggestion accuracy, improved user satisfaction, and enhanced system operational efficiency. Specific application fields include personal health management apps, lifestyle disease prevention programs at medical institutions, corporate health management support services, and health promotion programs by insurance companies.

The suggestion unit can refer to the user's past diet history and make optimal suggestions at the time of suggestion. For example, the suggestion unit refers to the user's past diet history and makes optimal suggestions at the time of suggestion. For instance, the suggestion unit makes suggestions by referring to diet methods that the user has succeeded with in the past. The suggestion unit can also make suggestions to avoid diet methods that the user has failed with in the past. Furthermore, the suggestion unit can customize the content of suggestions based on the user's past diet history. By referring to the user's past diet history, more effective suggestions become possible. Specifically, the suggestion unit acquires diet history data recorded in chronological order for each user (e.g., weight transition vectors over the past 5 years, start/end dates, target weight, actual weight, achievement status, labels of diet methods used for each diet period, weekly exercise amount, meal content, physical condition records as structured data) from a database. The suggestion unit performs feature extraction on these history data (e.g., goal achievement rate, average weight loss speed, presence of rebound, success rate for each diet method, labeling of failure factors), and uses machine learning models (e.g., decision trees, random forests, gradient boosting, clustering algorithms) or rule-based algorithms to automatically analyze past success and failure patterns of the user. Examples of AI inputs include (1) time-series vectors of weight transition (e.g., real values for 365 days), (2) diet method category label arrays (e.g., “low-carb”, “aerobic exercise”, “fasting”), and (3) structured data of goals, achievements, and achievement status for each period (e.g., JSON format). Examples of AI outputs include (1) recommended suggestion method labels (e.g., “gradual weight loss”, “short-term intensive”), (2) recommended goal achievement period (e.g., 90 days, 180 days), (3) recommended diet methods (e.g., “aerobic exercise+balanced diet”), and (4) failure risk score (e.g., 0.25). For example, if there are many successful records with “low-carb+aerobic exercise” in the past, the same method is recommended, and if there are many failures with “fasting”, that method is excluded. Furthermore, based on the AI output results, the suggestion unit performs subsequent processing such as automatic input of initial values on the suggestion screen, generation of suggestion advice messages to users, and automatic adjustment of suggestion content. Unlike conventional subjective counseling by humans or simple reference to history, the suggestion unit autonomously extracts features and recognizes patterns from high-dimensional history data using AI, thereby realizing automatic suggestion of optimized suggestion methods for each user. For AI model training, classification and regression tasks using past diet history and goal achievement status as training data, error backpropagation using cross-entropy loss or mean squared error as the loss function, and data augmentation (e.g., time-series shifting of history data, sampling of method labels) are utilized to enhance model generalization. As a technical effect, the suggestion unit can analyze the user's past behavior and outcome data with high accuracy and automate individually optimized, realistic suggestions, thereby achieving improved goal achievement rate, prevention of rebound, improved user satisfaction, and enhanced system operational efficiency. Specific application fields include personal diet apps, lifestyle disease prevention programs at medical institutions, corporate health management support services, and personal training support systems.

The suggestion unit can estimate a user's emotion and determine the priority of suggestions based on the estimated emotion. For example, the suggestion unit estimates the user's emotion and determines the priority of suggestions according to the estimated emotion. For instance, when the user is highly motivated, the suggestion unit prioritizes exercise plans. When the user is stressed, the suggestion unit can prioritize meal plans. Furthermore, when the user is relaxed, the suggestion unit can make suggestions considering overall balance. By determining the priority of suggestions according to the user's emotion, more effective suggestions become possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the suggestion unit inputs natural language text obtained from the user (e.g., comments when entering diet goals, diaries, survey responses of 50 to 500 tokens), audio data (e.g., 1-minute audio waveform, 16 kHz sampling), and facial or expression images (e.g., 3×224×224 pixels) into an emotion estimation AI model. The suggestion unit performs preprocessing on the input data (tokenization of text, spectrogram conversion of audio, extraction of facial regions from images) and generates feature vectors (e.g., text embedding vectors, audio feature vectors, image feature vectors). The suggestion unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The suggestion unit obtains outputs from the AI model such as emotion labels (e.g., “high motivation”, “stress”, “relaxation”), emotion scores (e.g., motivation level 0.85, stress level 0.60, relaxation level 0.40). For example, when text such as “I'm motivated this week”, a cheerful voice, and a smiling image are input, the output may be “motivation level 0.90”. The suggestion unit uses these emotion scores for threshold judgment and rule-based branching processing, and automatically determines the priority of suggestions (e.g., prioritize exercise plans, prioritize meal plans, balanced suggestions). For example, when motivation level is high, exercise plans are prioritized; when stress level is high, meal plans are prioritized, and so on. The suggestion unit uses the priority decision results for initial display on the suggestion screen, generation of advice messages to users, and automatic adjustment of suggestion plans. Unlike conventional subjective judgment by humans or simple selection of suggestion items, the suggestion unit realizes user-optimized suggestion priorities by combining multimodal emotion estimation in high-dimensional feature space and rule-based automatic priority determination. For AI model training, supervised learning using emotion-annotated datasets, error backpropagation using cross-entropy loss or mean squared error as the loss function, and data augmentation (e.g., text paraphrase generation, audio pitch conversion, image rotation) are utilized to enhance model generalization. As a technical effect, the suggestion unit can grasp the user's emotional state in real time and with high accuracy, greatly improving the realism and appropriateness of suggestion priorities, thereby achieving improved user experience, improved suggestion execution rate, and enhanced system operational efficiency. Specific application fields include personal health management apps, lifestyle disease prevention programs at medical institutions, corporate health management support services, and mental health care support systems.

The suggestion unit can make optimal suggestions by considering the user's geographic location information at the time of suggestion. For example, the suggestion unit makes optimal suggestions by considering the user's geographic location information at the time of suggestion. For instance, the suggestion unit proposes meal plans by considering local ingredients available in the user's region. The suggestion unit can also propose exercise plans by considering exercise facilities that are easily accessible to the user. Furthermore, the suggestion unit can propose exercise plans by considering the climate of the region where the user lives. By considering the user's geographic location information, more realistic suggestions become possible. Specifically, the suggestion unit acquires the user's location information data (e.g., GPS coordinates, prefecture/city labels, latitude/longitude vectors), regional climate data (e.g., monthly average temperature, precipitation, snowfall as time-series vectors), surrounding facility data (e.g., list of gyms, exercise facilities, parks within a 2 km radius), and local ingredient data (e.g., list of ingredients handled by local supermarkets, seasonal ingredient labels) from databases or external APIs and inputs them as structured data into the AI model. The suggestion unit performs preprocessing on these data (e.g., geocoding of location information, time-series aggregation of climate data, categorization of facility data), feature extraction (e.g., exercise facility accessibility score, climate adaptability score, local ingredient diversity score), and uses machine learning models (e.g., random forest, neural network) or rule-based algorithms to optimize the content of suggestions according to the user's geographic location information. Examples of AI inputs include (1) GPS coordinate vectors (e.g., latitude 35.6, longitude 139.7), (2) regional climate vectors (e.g., monthly average temperature 20° C., precipitation 100 mm), (3) surrounding facility category arrays (e.g., “2 gyms”, “1 park”), and (4) local ingredient label arrays (e.g., “komatsuna”, “mackerel”). Examples of AI outputs include (1) recommended meal plans (e.g., recipes using local ingredients), (2) recommended exercise plans (e.g., “indoor aerobic exercise focused”), (3) recommended target deadlines (e.g., set longer in winter), and (4) facility usage recommendation labels (e.g., “recommend using nearest gym”). For example, in regions with heavy snowfall in winter, suggestions focused on indoor exercise are made, and in areas with many nearby gyms, suggestions recommending gym usage are made. Furthermore, based on the AI output results, the suggestion unit performs subsequent processing such as automatic input of initial values on the suggestion screen, generation of region-specific advice messages to users, and automatic adjustment of suggestion content. Unlike conventional subjective judgment by humans or simple selection of suggestion items, the suggestion unit realizes realistic and highly feasible suggestions for each user by combining integrated analysis of multidimensional geographic and regional characteristic data with rule-based automatic optimization. For AI model training, classification and regression tasks using geographic information, climate, facility data, and suggestion outcome data as training data, error backpropagation using cross-entropy loss or mean squared error as the loss function, and data augmentation (e.g., clustering of location information, adding noise to climate data) are utilized to enhance model generalization. As a technical effect, the suggestion unit automatically considers the user's geographic constraints and regional characteristics, and realizes highly accurate, realistic, and feasible suggestions, thereby achieving improved suggestion execution rate, improved user satisfaction, and enhanced system operational efficiency. Specific application fields include personal health management apps, lifestyle disease prevention programs at medical institutions, corporate health management support services, and region-specific health support systems.

The suggestion unit can analyze the user's social media activity and make relevant suggestions at the time of suggestion. For example, the suggestion unit analyzes the user's social media activity and makes relevant suggestions at the time of suggestion. For instance, the suggestion unit refers to meal content shared by the user on social media. The suggestion unit can also refer to exercise methods of influencers followed by the user. Furthermore, the suggestion unit can refer to suggestions from diet communities in which the user participates. By analyzing the user's social media activity, more relevant suggestions become possible. Specifically, the suggestion unit acquires the user's social media post data (e.g., post texts, images, videos, post dates, hashtags, number of likes/comments over the past year as structured data), follow relationship data (e.g., list of followed influencers, community participation history), and shared goal data (e.g., diet goals declared on SNS, progress report posts) via API and inputs them as structured data into the AI model. The suggestion unit performs preprocessing on these data (e.g., tokenization of text, feature extraction from images, categorization of hashtags), feature extraction (e.g., frequency of diet-related posts, influencer recommendation method score, community goal distribution), and uses machine learning models (e.g., transformer-based language models, image classification models, clustering algorithms) or rule-based algorithms to optimize the content of suggestions based on the user's social media activity. Examples of AI inputs include (1) post text vectors (e.g., embedding vectors for 100 posts), (2) followed influencer label arrays (e.g., “muscle training”, “low-carb”), and (3) community goal label arrays (e.g., “lose 5 kg in 3 months”). Examples of AI outputs include (1) recommended meal plans (e.g., recipes referencing SNS shared meal images), (2) recommended exercise plans (e.g., influencer recommended exercise methods), (3) community goal reference labels (e.g., “conform to group goal”), and (4) recommended suggestion frequency (e.g., high frequency during community events). For example, if the user declares “lose 5 kg in 3 months” on SNS, the same suggestion is automatically set, and if a followed influencer recommends “aerobic exercise+high-protein diet”, that suggestion is prioritized. Furthermore, based on the AI output results, the suggestion unit performs subsequent processing such as automatic input of initial values on the suggestion screen, generation of SNS cooperation advice messages to users, and automatic adjustment of suggestion plans. Unlike conventional subjective judgment by humans or simple selection of suggestion items, the suggestion unit realizes highly relevant suggestions for each user by combining integrated analysis of multidimensional social media data with rule-based automatic suggestion setting. For AI model training, classification and regression tasks using SNS post data and suggestion outcome data as training data, error backpropagation using cross-entropy loss or mean squared error as the loss function, and data augmentation (e.g., paraphrase generation of post texts, image rotation/scaling) are utilized to enhance model generalization. As a technical effect, the suggestion unit automatically analyzes the user's social media activity and realizes highly accurate, relevant suggestions, thereby achieving improved suggestion accuracy, improved user satisfaction, and enhanced system operational efficiency. Specific application fields include personal health management apps, SNS-linked health support services, corporate health management support services, and community-based diet support systems.

The modification unit can estimate a user's emotion and adjust the content of modifications based on the estimated emotion. For example, the modification unit estimates the user's emotion and adjusts the content of modifications according to the estimated emotion. For instance, when the user is relaxed, the modification unit proposes detailed modifications. When the user is stressed, the modification unit can propose concise modifications. Furthermore, when the user is highly motivated, the modification unit can propose rigorous modifications. By adjusting the content of modifications according to the user's emotion, more appropriate modifications become possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the modification unit inputs natural language text obtained from the user (e.g., diet diary comments, meal record comments, survey responses of 50 to 500 tokens), audio data (e.g., 1-minute audio waveform, 16 kHz sampling), and facial or expression images (e.g., 3×224×224 pixels) into an emotion estimation AI model. The modification unit performs preprocessing on the input data (tokenization of text, spectrogram conversion of audio, extraction of facial regions from images) and generates feature vectors (e.g., text embedding vectors, audio feature vectors, image feature vectors). The modification unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The modification unit obtains outputs from the AI model such as emotion labels (e.g., “relaxed”, “stressed”, “high motivation”), emotion scores (e.g., stress level 0.72, relaxation level 0.85), and emotion change trends (e.g., transition graph over the past 7 days). For example, when text such as “I feel good today”, a calm voice, and a smiling image are input, the output may be “relaxation level 0.90”, “stress level 0.10”. In another example, when text such as “I've been busy and tired lately”, a depressed voice, and an expressionless image are input, the output may be “stress level 0.80”, “relaxation level 0.20”. The modification unit uses these emotion scores for threshold judgment (e.g., detailed modification if relaxation level is 0.7 or higher, concise modification if stress level is 0.6 or higher, rigorous modification if motivation level is 0.8 or higher) and rule-based branching processing, and automatically switches the granularity of modifications (e.g., modifications with detailed explanations, concise modifications with only key points, rigorous modifications emphasizing numerical evidence). The modification unit reflects the modification content in the user interface and uses it for subsequent processing such as generating advice messages to users and automatic summarization of modification content. Unlike conventional uniform modification display or manual selection of expression methods, the modification unit realizes user-optimized modification content by combining multimodal emotion estimation in high-dimensional feature space and rule-based automatic modification optimization. For AI model training, supervised learning using emotion-annotated datasets, error backpropagation using cross-entropy loss or mean squared error as the loss function, and data augmentation (e.g., text paraphrase generation, audio pitch conversion, image rotation) are utilized to enhance model generalization. As a technical effect, the modification unit can grasp the user's emotional state in real time and with high accuracy, greatly improving the realism and appropriateness of modification content, thereby achieving improved user experience, improved modification execution rate, and enhanced system operational efficiency. Specific application fields include personal health management apps, lifestyle disease prevention programs at medical institutions, corporate health management support services, and mental health care support systems.

The modification unit can customize the content of modifications based on the user's health status and lifestyle habits at the time of modification. For example, the modification unit customizes the content of modifications based on the user's health status and lifestyle habits at the time of modification. For instance, the modification unit customizes the content of modifications by referring to the user's health checkup results. The modification unit can also customize the content of modifications by considering the user's lifestyle habits (diet, exercise, sleep). Furthermore, the modification unit can customize the content of modifications by considering the user's medical history. By customizing the content of modifications based on the user's health status and lifestyle habits, more effective modifications become possible. Specifically, the modification unit acquires health checkup data obtained from the user (e.g., height, weight, BMI, blood pressure, blood glucose, cholesterol, liver function values as numerical vectors), lifestyle habit data (e.g., one week of meal records, exercise frequency, average sleep time, presence or absence of drinking/smoking habits), and medical history data (e.g., past disease labels, treatment history, medication information) from a database and inputs them as structured data into the AI model. The modification unit performs preprocessing on these data (e.g., missing value imputation, normalization, category conversion), feature extraction (e.g., health risk score, lifestyle habit score, disease risk label), and uses machine learning models (e.g., random forest, logistic regression, neural network) or rule-based algorithms to customize the content of modifications according to the user's health status and lifestyle habits. Examples of AI inputs include (1) health checkup numerical vectors (e.g., 10 dimensions), (2) lifestyle habit category label arrays (e.g., “skipping breakfast”, “exercise twice a week”, “6 hours sleep”), and (3) medical history label arrays (e.g., “hypertension”, “diabetes”). Examples of AI outputs include (1) recommended modification content labels (e.g., “modification to low-salt Japanese cuisine”), (2) recommended exercise plan modification (e.g., “change to light aerobic exercise”), (3) risk warning labels (e.g., “rapid weight loss not allowed”), and (4) recommended lifestyle improvement actions (e.g., “extend sleep time”). For example, if hypertension is found in a health checkup, modifications to low-salt meals and light exercise are recommended, and if there is a tendency for sleep deprivation, modifications prioritizing sleep improvement are made. Furthermore, based on the AI output results, the modification unit performs subsequent processing such as input restrictions on the modification screen (e.g., automatic blocking of dangerous modifications), display of health risk warnings to users, and automatic adjustment of modification content. Unlike conventional subjective advice by humans or simple modifications, the modification unit realizes safe and feasible modifications for each user by combining integrated analysis of multidimensional health and lifestyle habit data with rule-based automatic customization. For AI model training, classification and regression tasks using health checkup data and modification outcome data as training data, error backpropagation using cross-entropy loss or mean squared error as the loss function, and data augmentation (e.g., adding noise to health checkup values, sampling lifestyle habit patterns) are utilized to enhance model generalization. As a technical effect, the modification unit automatically avoids health risks for users and realizes highly accurate, realistic, and safe modifications, thereby achieving reduced risk of health damage, improved modification accuracy, improved user satisfaction, and enhanced system operational efficiency. Specific application fields include personal health management apps, lifestyle disease prevention programs at medical institutions, corporate health management support services, and health promotion programs by insurance companies.

The modification unit can refer to the user's past diet history and make optimal modifications at the time of modification. For example, the modification unit refers to the user's past diet history and makes optimal modifications at the time of modification. For instance, the modification unit makes modifications by referring to diet methods that the user has succeeded with in the past. The modification unit can also make modifications to avoid diet methods that the user has failed with in the past. Furthermore, the modification unit can customize the content of modifications based on the user's past diet history. By referring to the user's past diet history, more effective modifications become possible. Specifically, the modification unit acquires diet history data recorded in chronological order for each user (e.g., weight transition vectors over the past 5 years, start/end dates, target weight, actual weight, achievement status, labels of diet methods used for each diet period, weekly exercise amount, meal content, physical condition records as structured data) from a database. The modification unit performs feature extraction on these history data (e.g., goal achievement rate, average weight loss speed, presence of rebound, success rate for each diet method, labeling of failure factors), and uses machine learning models (e.g., decision trees, random forests, gradient boosting, clustering algorithms) or rule-based algorithms to automatically analyze past success and failure patterns of the user. Examples of AI inputs include (1) time-series vectors of weight transition (e.g., real values for 365 days), (2) diet method category label arrays (e.g., “low-carb”, “aerobic exercise”, “fasting”), and (3) structured data of goals, achievements, and achievement status for each period (e.g., JSON format). Examples of AI outputs include (1) recommended modification method labels (e.g., “modification to gradual weight loss”, “modification to short-term intensive”), (2) recommended goal achievement period modification (e.g., 90 days, 180 days), (3) recommended diet method modification (e.g., “aerobic exercise+balanced diet”), and (4) failure risk score (e.g., 0.25). For example, if there are many successful records with “low-carb+aerobic exercise” in the past, modification to the same method is recommended, and if there are many failures with “fasting”, that method is excluded. Furthermore, based on the AI output results, the modification unit performs subsequent processing such as automatic input of initial values on the modification screen, generation of modification advice messages to users, and automatic adjustment of modification content. Unlike conventional subjective counseling by humans or simple reference to history, the modification unit autonomously extracts features and recognizes patterns from high-dimensional history data using AI, thereby realizing automatic proposal of optimized modification methods for each user. For AI model training, classification and regression tasks using past diet history and goal achievement status as training data, error backpropagation using cross-entropy loss or mean squared error as the loss function, and data augmentation (e.g., time-series shifting of history data, sampling of method labels) are utilized to enhance model generalization. As a technical effect, the modification unit can analyze the user's past behavior and outcome data with high accuracy and automate individually optimized, realistic modifications, thereby achieving improved goal achievement rate, prevention of rebound, improved user satisfaction, and enhanced system operational efficiency. Specific application fields include personal diet apps, lifestyle disease prevention programs at medical institutions, corporate health management support services, and personal training support systems.

The modification unit can estimate a user's emotion and determine the priority of modifications based on the estimated emotion. For example, the modification unit estimates the user's emotion and determines the priority of modifications according to the estimated emotion. For instance, when the user is highly motivated, the modification unit prioritizes modification of exercise plans. When the user is stressed, the modification unit can prioritize modification of meal plans. Furthermore, when the user is relaxed, the modification unit can make modifications considering overall balance. By determining the priority of modifications according to the user's emotion, more effective modifications become possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the modification unit inputs natural language text obtained from the user (e.g., diet diary comments, meal record comments, survey responses of 50 to 500 tokens), audio data (e.g., 1-minute audio waveform, 16 kHz sampling), and facial or expression images (e.g., 3×224×224 pixels) into an emotion estimation AI model. The modification unit performs preprocessing on the input data (tokenization of text, spectrogram conversion of audio, extraction of facial regions from images) and generates feature vectors (e.g., text embedding vectors, audio feature vectors, image feature vectors). The modification unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The modification unit obtains outputs from the AI model such as emotion labels (e.g., “high motivation”, “stress”, “relaxation”), emotion scores (e.g., motivation level 0.85, stress level 0.60, relaxation level 0.40). For example, when text such as “I'm motivated this week”, a cheerful voice, and a smiling image are input, the output may be “motivation level 0.90”. The modification unit uses these emotion scores for threshold judgment and rule-based branching processing, and automatically determines the priority of modifications (e.g., prioritize modification of exercise plans, prioritize modification of meal plans, balanced modifications). For example, when motivation level is high, modification of exercise plans is prioritized; when stress level is high, modification of meal plans is prioritized, and so on. The modification unit uses the priority decision results for initial display on the modification screen, generation of advice messages to users, and automatic adjustment of modification plans. Unlike conventional subjective judgment by humans or simple selection of modification items, the modification unit realizes user-optimized modification priorities by combining multimodal emotion estimation in high-dimensional feature space and rule-based automatic priority determination. For AI model training, supervised learning using emotion-annotated datasets, error backpropagation using cross-entropy loss or mean squared error as the loss function, and data augmentation (e.g., text paraphrase generation, audio pitch conversion, image rotation) are utilized to enhance model generalization. As a technical effect, the modification unit can grasp the user's emotional state in real time and with high accuracy, greatly improving the realism and appropriateness of modification priorities, thereby achieving improved user experience, improved modification execution rate, and enhanced system operational efficiency. Specific application fields include personal health management apps, lifestyle disease prevention programs at medical institutions, corporate health management support services, and mental health care support systems.

The modification unit can make optimal modifications by considering the user's geographic location information at the time of modification. For example, the modification unit makes optimal modifications by considering the user's geographic location information at the time of modification. For instance, the modification unit modifies meal plans by considering local ingredients available in the user's region. The modification unit can also modify exercise plans by considering exercise facilities that are easily accessible to the user. Furthermore, the modification unit can modify exercise plans by considering the climate of the region where the user lives. By considering the user's geographic location information, more realistic modifications become possible. Specifically, the modification unit acquires the user's location information data (e.g., GPS coordinates, prefecture/city labels, latitude/longitude vectors), regional climate data (e.g., monthly average temperature, precipitation, snowfall as time-series vectors), surrounding facility data (e.g., list of gyms, exercise facilities, parks within a 2 km radius), and local ingredient data (e.g., list of ingredients handled by local supermarkets, seasonal ingredient labels) from databases or external APIs and inputs them as structured data into the AI model. The modification unit performs preprocessing on these data (e.g., geocoding of location information, time-series aggregation of climate data, categorization of facility data), feature extraction (e.g., exercise facility accessibility score, climate adaptability score, local ingredient diversity score), and uses machine learning models (e.g., random forest, neural network) or rule-based algorithms to optimize the content of modifications according to the user's geographic location information. Examples of AI inputs include (1) GPS coordinate vectors (e.g., latitude 35.6, longitude 139.7), (2) regional climate vectors (e.g., monthly average temperature 20° C., precipitation 100 mm), (3) surrounding facility category arrays (e.g., “2 gyms”, “1 park”), and (4) local ingredient label arrays (e.g., “komatsuna”, “mackerel”). Examples of AI outputs include (1) recommended modification of meal plans (e.g., modification to recipes using local ingredients), (2) recommended modification of exercise plans (e.g., modification to “indoor aerobic exercise focused”), (3) recommended modification of target deadlines (e.g., set longer in winter), and (4) facility usage recommendation labels (e.g., “recommend using nearest gym”). For example, in regions with heavy snowfall in winter, modifications focused on indoor exercise are made, and in areas with many nearby gyms, modifications recommending gym usage are made. Furthermore, based on the AI output results, the modification unit performs subsequent processing such as automatic input of initial values on the modification screen, generation of region-specific advice messages to users, and automatic adjustment of modification content. Unlike conventional subjective judgment by humans or simple selection of modification items, the modification unit realizes realistic and highly feasible modifications for each user by combining integrated analysis of multidimensional geographic and regional characteristic data with rule-based automatic optimization. For AI model training, classification and regression tasks using geographic information, climate, facility data, and modification outcome data as training data, error backpropagation using cross-entropy loss or mean squared error as the loss function, and data augmentation (e.g., clustering of location information, adding noise to climate data) are utilized to enhance model generalization. As a technical effect, the modification unit automatically considers the user's geographic constraints and regional characteristics, and realizes highly accurate, realistic, and feasible modifications, thereby achieving improved modification execution rate, improved user satisfaction, and enhanced system operational efficiency. Specific application fields include personal health management apps, lifestyle disease prevention programs at medical institutions, corporate health management support services, and region-specific health support systems.

The modification unit can analyze the user's social media activity and make relevant modifications at the time of modification. For example, the modification unit analyzes the user's social media activity and makes relevant modifications at the time of modification. For instance, the modification unit refers to meal content shared by the user on social media. The modification unit can also refer to exercise methods of influencers followed by the user. Furthermore, the modification unit can refer to modifications from diet communities in which the user participates. By analyzing the user's social media activity, more relevant modifications become possible. Specifically, the modification unit acquires the user's social media post data (e.g., post texts, images, videos, post dates, hashtags, number of likes/comments over the past year as structured data), follow relationship data (e.g., list of followed influencers, community participation history), and shared goal data (e.g., diet goals declared on SNS, progress report posts) via API and inputs them as structured data into the AI model. The modification unit performs preprocessing on these data (e.g., tokenization of text, feature extraction from images, categorization of hashtags), feature extraction (e.g., frequency of diet-related posts, influencer recommendation method score, community goal distribution), and uses machine learning models (e.g., transformer-based language models, image classification models, clustering algorithms) or rule-based algorithms to optimize the content of modifications based on the user's social media activity. Examples of AI inputs include (1) post text vectors (e.g., embedding vectors for 100 posts), (2) followed influencer label arrays (e.g., “muscle training”, “low-carb”), and (3) community goal label arrays (e.g., “lose 5 kg in 3 months”). Examples of AI outputs include (1) recommended modification of meal plans (e.g., modification to recipes referencing SNS shared meal images), (2) recommended modification of exercise plans (e.g., modification to influencer recommended exercise methods), (3) community goal reference labels (e.g., “conform to group goal”), and (4) recommended modification frequency (e.g., high frequency during community events). For example, if the user declares “lose 5 kg in 3 months” on SNS, the same modification is automatically set, and if a followed influencer recommends “aerobic exercise+high-protein diet”, that modification is prioritized. Furthermore, based on the AI output results, the modification unit performs subsequent processing such as automatic input of initial values on the modification screen, generation of SNS cooperation advice messages to users, and automatic adjustment of modification plans. Unlike conventional subjective judgment by humans or simple selection of modification items, the modification unit realizes highly relevant modifications for each user by combining integrated analysis of multidimensional social media data with rule-based automatic modification setting. For AI model training, classification and regression tasks using SNS post data and modification outcome data as training data, error backpropagation using cross-entropy loss or mean squared error as the loss function, and data augmentation (e.g., paraphrase generation of post texts, image rotation/scaling) are utilized to enhance model generalization. As a technical effect, the modification unit automatically analyzes the user's social media activity and realizes highly accurate, relevant modifications, thereby achieving improved modification accuracy, improved user satisfaction, and enhanced system operational efficiency. Specific application fields include personal health management apps, SNS-linked health support services, corporate health management support services, and community-based diet support systems.

The image recognition unit can estimate a user's emotion and adjust the accuracy of image recognition based on the estimated emotion. For example, the image recognition unit estimates the user's emotion and adjusts the accuracy of image recognition according to the estimated emotion. For instance, when the user is relaxed, the image recognition unit performs detailed image recognition. When the user is stressed, the image recognition unit can perform concise image recognition. Furthermore, when the user is highly motivated, the image recognition unit can perform rigorous image recognition. By adjusting the accuracy of image recognition according to the user's emotion, more accurate image recognition becomes possible. Emotion estimation is realized, for example, by using an emotion estimation function implemented with an emotion engine or generative AI. Generative AI may include text generation AI (e.g., LLM) or multimodal generative AI, but is not limited to these examples. Specifically, the image recognition unit inputs natural language text obtained from the user (e.g., meal record comments, diaries, survey responses of 50 to 500 tokens), audio data (e.g., 1-minute audio waveform, 16 kHz sampling), and facial or expression images (e.g., 3×224×224 pixels) into an emotion estimation AI model. The image recognition unit performs preprocessing on the input data (tokenization of text, spectrogram conversion of audio, extraction of facial regions from images) and generates feature vectors (e.g., text embedding vectors, audio feature vectors, image feature vectors). The image recognition unit uses a multimodal emotion estimation model that combines transformer-based large language models (e.g., 12 layers, 768-dimensional hidden layers), convolutional neural networks (e.g., ResNet-18), and recurrent neural networks for audio emotion classification (e.g., 2-layer LSTM). The image recognition unit obtains outputs from the AI model such as emotion labels (e.g., “relaxed”, “stressed”, “high motivation”), emotion scores (e.g., stress level 0.72, relaxation level 0.85), and emotion change trends (e.g., transition graph over the past 7 days). For example, when text such as “I feel good today”, a calm voice, and a smiling image are input, the output may be “relaxation level 0.90”, “stress level 0.10”. In another example, when text such as “I've been busy and tired lately”, a depressed voice, and an expressionless image are input, the output may be “stress level 0.80”, “relaxation level 0.20”. The image recognition unit uses these emotion scores for threshold judgment (e.g., detailed recognition if relaxation level is 0.7 or higher, simple recognition if stress level is 0.6 or higher, rigorous recognition if motivation level is 0.8 or higher) and rule-based branching processing, and automatically switches the image recognition method (e.g., number of layers in the recognition model, selection of features, recognition resolution, recognition granularity). For example, detailed recognition uses multilayer neural network analysis with all features, simple recognition uses decision tree analysis with only major features, and rigorous recognition applies ensemble learning or Bayesian optimization. The image recognition unit uses image inputs such as meal images (e.g., 3×224×224 pixel RGB images), exercise scene images (e.g., 3×224×224 pixels), and food package images (e.g., 3×224×224 pixels). Examples of AI outputs include meal content labels (e.g., “salad”, “grilled fish”, “rice”), ingredient recognition scores (e.g., salad 0.92, rice 0.85), estimated intake amounts (e.g., rice 150 g), and recognition confidence (e.g., 0.95). For example, when an image of grilled salmon and rice is input, the output may be “grilled fish 0.90, rice 0.88”. Furthermore, based on the AI output results, the image recognition unit performs subsequent processing such as automatic recording of recognition results, generation of feedback messages to users, and automatic adjustment of recognition accuracy. Unlike conventional subjective recognition by humans or simple image classification, the image recognition unit realizes user-optimized image recognition by combining multimodal emotion estimation in high-dimensional feature space and rule-based automatic optimization of recognition methods. For AI model training, classification and regression tasks using emotion-annotated datasets and image recognition outcome data as training data, error backpropagation using cross-entropy loss or mean squared error as the loss function, and data augmentation (e.g., image rotation/scaling, text paraphrase generation, audio pitch conversion) are utilized to enhance model generalization and robustness. As a technical effect, the image recognition unit can grasp the user's emotional state in real time and with high accuracy, greatly improving the realism, accuracy, and efficiency of image recognition methods, thereby achieving improved image recognition accuracy, reduced user burden, and enhanced operational efficiency of the entire system. Specific application fields include personal meal management apps, meal record support at medical institutions, corporate health management support services, and automatic food intake recording systems.

The image recognition unit can optimize the recognition algorithm by referring to past image data during image recognition. For example, the image recognition unit optimizes the recognition algorithm by referring to past image data during image recognition. For instance, the image recognition unit optimizes the recognition algorithm by referring to past successful cases. The image recognition unit can also optimize the recognition algorithm to avoid past failure cases. Furthermore, the image recognition unit can propose optimal recognition methods based on past image recognition history. By referring to past image data, optimization of the recognition algorithm becomes possible. Specifically, the image recognition unit acquires past image recognition history data recorded in chronological order for each user (e.g., recognition result history of meal images, exercise images, food images over the past year, input image feature vectors for each recognition, parameters of used models, accuracy indicators of recognition results, user feedback scores, error codes in case of failure, etc.) from a database. The image recognition unit performs feature extraction on these history data (e.g., accuracy trends for each model, labeling of failure factors, recognition success rate, time-series changes in user satisfaction scores), and uses machine learning models (e.g., decision trees, random forests, Bayesian optimization algorithms, meta-learning models) or rule-based algorithms to automatically analyze past success and failure patterns. Examples of AI inputs include (1) time-series vectors of image recognition history (e.g., model ID, accuracy, failure flag for 365 days), (2) input image feature vectors (e.g., image embedding vectors), (3) user feedback scores (e.g., 0.8, 0.6, 0.9), and (4) error code arrays (e.g., 0=success, 1=blurry image, 2=model mismatch). Examples of AI outputs include (1) recommended recognition algorithm labels (e.g., “ResNet-50”, “EfficientNet”, “ensemble learning”), (2) recommended hyperparameters (e.g., learning rate 0.01, number of layers 4), (3) failure risk score (e.g., 0.12), and (4) recommended recognition frequency (e.g., once per day). For example, if high-accuracy recognition results were obtained in the past using “ResNet-50”, the same model is recommended, and if there were many failures with “decision tree”, that method is excluded. Furthermore, based on the AI output results, the image recognition unit performs subsequent processing such as automatic switching of recognition algorithms, parameter optimization, automatic adjustment of recognition frequency, and generation of explanatory messages for recognition methods to users. Unlike conventional subjective model selection by humans or simple application of fixed algorithms, the image recognition unit autonomously extracts features and recognizes patterns from high-dimensional history data using AI, thereby realizing automatic selection and optimization of recognition algorithms optimized for each user. For AI model training, classification and regression tasks using past image recognition history and recognition accuracy/failure as training data, error backpropagation using cross-entropy loss or mean squared error as the loss function, and data augmentation (e.g., time-series shifting of history data, sampling of model parameters) are utilized to enhance model generalization and robustness. As a technical effect, the image recognition unit can analyze the user's past image recognition history with high accuracy and realize automatic selection and optimization of individually optimized recognition algorithms, thereby achieving improved recognition accuracy, reduced failure rate, improved user satisfaction, and enhanced system operational efficiency. Specific application fields include personal meal management apps, meal record support at medical institutions, corporate health management support services, and automatic food intake recording systems.

The image recognition unit can improve recognition accuracy during image recognition based on the user's meal history. For example, the image recognition unit improves recognition accuracy during image recognition by referring to the user's meal history. Specifically, the image recognition unit performs image recognition by referencing the user's past meal history, and can also consider the user's meal patterns during image recognition. Furthermore, the image recognition unit can propose an optimal recognition method based on the user's meal history. By improving recognition accuracy based on the user's meal history, more accurate image recognition becomes possible. Specifically, the image recognition unit acquires meal history data recorded in chronological order for each user (e.g., an array of meal content labels for the past year, intake amount vectors, meal time zones, ingredient categories, preference labels, and other structured data) from a database. The image recognition unit performs feature extraction on these history data (e.g., ranking of frequently consumed ingredients, clustering of meal patterns, time-series changes in intake amounts, preference tendency scores), and uses machine learning models (e.g., clustering algorithms, Bayesian optimization, neural networks) or rule-based algorithms to select recognition methods and optimize recognition parameters according to the user's meal history. As examples of input to AI, the image recognition unit uses (1) meal history label arrays (e.g., “rice,” “salad,” “grilled fish” for 365 days), (2) intake amount vectors (e.g., grams per ingredient), (3) meal time zone arrays (e.g., “morning,” “afternoon,” “evening”), and (4) preference score vectors (e.g., Japanese cuisine 0.8, Western cuisine 0.2). As examples of AI output, the image recognition unit generates (1) recommended recognition model labels (e.g., “Japanese cuisine-specialized CNN,” “multi-ingredient compatible ResNet”), (2) recommended recognition parameters (e.g., feature selection lists, recognition resolution), (3) recognition accuracy scores (e.g., 0.94), and (4) candidate ingredient lists (e.g., “rice,” “miso soup,” “grilled fish”). For example, if the meal history is mainly Japanese cuisine, a Japanese cuisine-specialized model is automatically selected; if Western cuisine is predominant, a Western cuisine-specialized model is selected. Furthermore, based on the AI output, the image recognition unit performs subsequent processing such as automatic switching of recognition methods, optimization of recognition parameters, display of advice to improve recognition accuracy to the user, and reflection of recognition results in the history. Unlike conventional subjective selection of recognition methods by humans or simple uniform recognition, the image recognition unit realizes highly accurate and practical image recognition for each user by combining integrated analysis of multidimensional meal history data and rule-based automatic optimization. For AI model training, classification and regression tasks are performed using meal history data and image recognition result data as training data, error backpropagation using loss functions such as cross-entropy loss and mean squared error, and data augmentation (e.g., adding noise to meal history, sampling ingredient categories) to enhance model generalization performance. As a technical effect, the image recognition unit automatically considers the user's meal tendencies and realizes practical and highly accurate image recognition, thereby improving recognition accuracy, user satisfaction, and system operation efficiency. Specific application fields include personal meal management apps, meal record support in medical institutions, corporate health management support services, and automatic food intake recording systems.

The image recognition unit can estimate the user's emotion and adjust the display method of image recognition results based on the estimated emotion. For example, the image recognition unit estimates the user's emotion and adjusts the display method of image recognition results according to the estimated emotion. For instance, if the user is nervous, the image recognition unit provides a simple and highly visible display method. If the user is relaxed, the image recognition unit can provide a display method that includes detailed information. Furthermore, if the user is in a hurry, the image recognition unit can provide a display method that emphasizes key points. By adjusting the display method of image recognition results according to the user's emotion, more appropriate information provision becomes possible. Emotion estimation is realized, for example, by using an emotion engine or generative AI with emotion estimation functions. Generative AI may include text generation AI (e.g., LLM) or multimodal generative AI, but is not limited to these examples. Specifically, the image recognition unit inputs natural language text obtained from the user (e.g., meal record comments, diaries, questionnaire responses of 50 to 500 tokens), audio data (e.g., one-minute audio waveform, 16 kHz sampling), and facial or expression images (e.g., 3×224×224 pixels) into an emotion estimation AI model. The image recognition unit performs preprocessing on the input data (tokenization of text, conversion of audio to spectrogram, extraction of facial regions from images) and generates feature vectors (e.g., text embedding vectors, audio feature vectors, image feature vectors). The image recognition unit uses a multimodal emotion estimation model combining transformer-based large language models (e.g., 12 layers, 768-dimensional hidden layers), convolutional neural networks (e.g., ResNet-18), and recurrent neural networks for audio emotion classification (e.g., 2-layer LSTM). As AI model output, the image recognition unit obtains emotion labels (e.g., “nervous,” “relaxed,” “in a hurry”), emotion scores (e.g., nervousness 0.80, relaxation 0.60, urgency 0.70), and emotion change trends (e.g., transition graph for the past 7 days). For example, if text such as “I am nervous today,” fast speech, and a tense facial image are input, the output may be “nervousness 0.85.” The image recognition unit uses these emotion scores for threshold judgment and rule-based branching to automatically determine the display method of image recognition results (e.g., simple display, detailed display, key point display). For example, if nervousness is high, a simple display with minimal graphs and numbers is selected; if relaxation is high, a detailed display with recognition graphs and explanatory text is added; if urgency is high, a display emphasizing only key points is selected. The image recognition unit reflects the display method decision in the user interface and uses it for subsequent processing such as generating advice messages for the user and automatic summarization of recognition results. Unlike conventional uniform information display or manual selection of display methods, the image recognition unit realizes optimized image recognition result display for each user by combining multimodal emotion estimation in high-dimensional feature space and rule-based automatic display optimization. For AI model training, classification and regression tasks are performed using emotion-annotated datasets and image recognition result data as training data, error backpropagation using loss functions such as cross-entropy loss and mean squared error, and data augmentation (e.g., text paraphrase generation, audio pitch conversion, image rotation) to enhance model generalization performance. As a technical effect, the image recognition unit can grasp the user's emotional state in real time and with high accuracy, greatly improving the realism and appropriateness of image recognition result display, thereby enhancing user experience, information transmission efficiency, and system operation efficiency. Specific application fields include personal meal management apps, meal record support in medical institutions, corporate health management support services, and automatic food intake recording systems.

The image recognition unit can improve recognition accuracy during image recognition by considering the user's geographic location information. For example, the image recognition unit improves recognition accuracy during image recognition by considering the user's geographic location information. For instance, the image recognition unit performs image recognition by considering ingredients available in the user's residential area. The image recognition unit can also perform image recognition by considering ingredients that are easily accessible to the user. Furthermore, the image recognition unit can perform image recognition by considering the food culture of the user's residential area. By considering the user's geographic location information, more accurate image recognition becomes possible. Specifically, the image recognition unit acquires user location data (e.g., GPS coordinates, prefecture/city labels, latitude/longitude vectors), regional ingredient data (e.g., local supermarket ingredient lists, seasonal ingredient labels), regional food culture data (e.g., representative dish labels by region, food culture categories), and accessible ingredient data (e.g., inventory information of nearby stores) from databases or external APIs and inputs them as structured data into the AI model. The image recognition unit performs preprocessing on these data (e.g., geocoding of location information, categorization of ingredient data, labeling of food culture data), feature extraction (e.g., regional ingredient frequency scores, food culture compatibility scores, accessibility scores), and uses machine learning models (e.g., random forest, neural networks) or rule-based algorithms to select recognition methods and optimize recognition parameters according to the user's geographic location information. As examples of input to AI, the image recognition unit uses (1) GPS coordinate vectors (e.g., latitude 35.6, longitude 139.7), (2) regional ingredient label arrays (e.g., “komatsuna,” “mackerel”), (3) regional food culture categories (e.g., “Japanese cuisine,” “Chinese cuisine”), and (4) accessible ingredient lists (e.g., “chicken,” “cabbage”). As examples of AI output, the image recognition unit generates (1) recommended recognition model labels (e.g., “region-specialized CNN”), (2) recommended recognition parameters (e.g., regional ingredient weighting), (3) recognition accuracy scores (e.g., 0.93), and (4) candidate ingredient lists (e.g., “komatsuna,” “mackerel”). For example, if the user lives in Hokkaido and salmon is in season, salmon recognition is enhanced; if the user lives in Okinawa and goya is common, goya recognition is enhanced. Furthermore, based on the AI output, the image recognition unit performs subsequent processing such as automatic switching of recognition methods, optimization of recognition parameters, generation of region-specific advice messages for the user, and reflection of regional characteristics in recognition results. Unlike conventional uniform recognition processing or manual consideration of regional characteristics, the image recognition unit realizes practical and highly accurate image recognition for each user by combining integrated analysis of multidimensional geographic information and regional characteristic data with rule-based automatic optimization. For AI model training, classification and regression tasks are performed using geographic information, ingredient, and food culture data and image recognition result data as training data, error backpropagation using loss functions such as cross-entropy loss and mean squared error, and data augmentation (e.g., clustering of location information, adding noise to ingredient data) to enhance model generalization performance. As a technical effect, the image recognition unit automatically considers the user's geographic constraints and regional characteristics, and realizes practical and highly feasible image recognition with high accuracy, thereby improving recognition accuracy, user satisfaction, and system operation efficiency. Specific application fields include personal meal management apps, meal record support in medical institutions, corporate health management support services, and region-specialized meal management systems.

The image recognition unit can analyze the user's social media activity during image recognition and recognize relevant images. For example, the image recognition unit analyzes the user's social media activity during image recognition and recognizes relevant images. For instance, the image recognition unit refers to meal content shared by the user on social media. The image recognition unit can also refer to meal content of influencers followed by the user. Furthermore, the image recognition unit can refer to images from diet communities in which the user participates. By analyzing the user's social media activity, more relevant image recognition becomes possible. Specifically, the image recognition unit acquires the user's social media post data (e.g., images posted over the past year, post text, post date and time, hashtags, number of likes and comments as structured data), follow relationship data (e.g., list of followed influencers, community participation history), and shared goal data (e.g., diet goals declared on SNS, progress report posts) via API and inputs them as structured data into the AI model. The image recognition unit performs preprocessing on these data (e.g., feature extraction from images, tokenization of text, categorization of hashtags), feature extraction (e.g., frequency of diet-related images, influencer-recommended meal scores, community goal distribution), and uses machine learning models (e.g., transformer-based language models, image classification models, clustering algorithms) or rule-based algorithms to select image recognition targets and optimize recognition methods based on the user's social media activity. As examples of input to AI, the image recognition unit uses (1) post image feature vectors (e.g., image embedding vectors for 100 posts), (2) followed influencer label arrays (e.g., “muscle training,” “low-carb”), and (3) community goal label arrays (e.g., “5 kg loss in 3 months”). As examples of AI output, the image recognition unit generates (1) recommended recognition image types (e.g., “SNS shared meal images,” “influencer-recommended meal images”), (2) recommended recognition frequency values (e.g., high frequency during community events), (3) community goal reference labels (e.g., “conforms to group goal”), and (4) recognition accuracy scores (e.g., 0.91). For example, if the user declares “5 kg weight loss in 3 months” on SNS, similar image recognition is automatically set; if a followed influencer recommends “high-protein meals,” recognition of such images is prioritized. Furthermore, based on the AI output, the image recognition unit performs subsequent processing such as automatic input of initial values on the recognition screen, generation of SNS cooperation advice messages for the user, and automatic adjustment of recognition plans. Unlike conventional subjective judgment by humans or simple selection of recognition items, the image recognition unit realizes highly relevant image recognition for each user by combining integrated analysis of multidimensional social media data and rule-based automatic recognition setting. For AI model training, classification and regression tasks are performed using SNS post data and image recognition result data as training data, error backpropagation using loss functions such as cross-entropy loss and mean squared error, and data augmentation (e.g., rotation and scaling of post images, text paraphrase generation) to enhance model generalization performance. As a technical effect, the image recognition unit automatically analyzes the user's social media activity and realizes highly relevant image recognition with high accuracy, thereby improving recognition accuracy, user satisfaction, and system operation efficiency. Specific application fields include personal meal management apps, SNS-linked health support services, corporate health management support services, and community-based diet support systems.

The cooperation unit can estimate the user's emotion and select cooperation devices based on the estimated emotion. For example, the cooperation unit estimates the user's emotion and selects cooperation devices according to the estimated emotion. For instance, if the user is relaxed, the cooperation unit proposes detailed cooperation devices. If the user is feeling stressed, the cooperation unit can propose simple cooperation devices. Furthermore, if the user is highly motivated, the cooperation unit can propose strict cooperation devices. By selecting cooperation devices according to the user's emotion, more appropriate device cooperation becomes possible. Emotion estimation is realized, for example, by using an emotion engine or generative AI with emotion estimation functions. Generative AI may include text generation AI (e.g., LLM) or multimodal generative AI, but is not limited to these examples.

The cooperation unit can optimize the cooperation algorithm during cooperation by referring to past cooperation data. For example, the cooperation unit optimizes the cooperation algorithm during cooperation by referring to past cooperation data. For instance, the cooperation unit optimizes the cooperation algorithm by referencing past successful cases. The cooperation unit can also optimize the cooperation algorithm to avoid past failures. Furthermore, the cooperation unit can propose optimal cooperation methods based on the history of past cooperation data. By referring to past cooperation data, optimization of the cooperation algorithm becomes possible.

The cooperation unit can customize the cooperation content during cooperation based on the user's health status and lifestyle habits. For example, the cooperation unit customizes the cooperation content during cooperation based on the user's health status and lifestyle habits. For instance, the cooperation unit customizes the cooperation content by referencing the user's health checkup results. The cooperation unit can also customize the cooperation content by considering the user's lifestyle habits (diet, exercise, sleep). Furthermore, the cooperation unit can customize the cooperation content by considering the user's medical history. By customizing the cooperation content based on the user's health status and lifestyle habits, more effective device cooperation becomes possible.

The cooperation unit can estimate the user's emotion and determine the priority of cooperation based on the estimated emotion. For example, the cooperation unit estimates the user's emotion and determines the priority of cooperation according to the estimated emotion. For instance, if the user is highly motivated, the cooperation unit prioritizes cooperation with exercise devices. If the user is feeling stressed, the cooperation unit can prioritize cooperation with meal devices. Furthermore, if the user is relaxed, the cooperation unit can cooperate by considering overall balance. By determining the priority of cooperation according to the user's emotion, more effective device cooperation becomes possible. Emotion estimation is realized, for example, by using an emotion engine or generative AI with emotion estimation functions. Generative AI may include text generation AI (e.g., LLM) or multimodal generative AI, but is not limited to these examples.

The cooperation unit can perform optimal cooperation during cooperation by considering the user's geographic location information. For example, the cooperation unit performs optimal cooperation during cooperation by considering the user's geographic location information. For instance, the cooperation unit cooperates by considering ingredients available in the user's residential area. The cooperation unit can also cooperate by considering exercise facilities that are easily accessible to the user. Furthermore, the cooperation unit can cooperate by considering the climate of the user's residential area. By considering the user's geographic location information, more realistic device cooperation becomes possible.

The cooperation unit can analyze the user's social media activity during cooperation and perform relevant cooperation. For example, the cooperation unit analyzes the user's social media activity during cooperation and performs relevant cooperation. For instance, the cooperation unit refers to meal content shared by the user on social media. The cooperation unit can also refer to exercise methods of influencers followed by the user. Furthermore, the cooperation unit can refer to cooperation in diet communities in which the user participates. By analyzing the user's social media activity, more relevant device cooperation becomes possible.

The security unit can estimate the user's emotion and adjust the data encryption method based on the estimated emotion. For example, the security unit estimates the user's emotion and adjusts the data encryption method according to the estimated emotion. For instance, if the user is relaxed, the security unit performs detailed encryption. If the user is feeling stressed, the security unit can perform simple encryption. Furthermore, if the user is highly motivated, the security unit can perform strict encryption. By adjusting the data encryption method according to the user's emotion, more appropriate data protection becomes possible. Emotion estimation is realized, for example, by using an emotion engine or generative AI with emotion estimation functions. Generative AI may include text generation AI (e.g., LLM) or multimodal generative AI, but is not limited to these examples.

The security unit can optimize the encryption algorithm during data encryption by referring to past security data. For example, the security unit optimizes the encryption algorithm during data encryption by referring to past security data. For instance, the security unit optimizes the encryption algorithm by referencing past successful cases. The security unit can also optimize the encryption algorithm to avoid past failures. Furthermore, the security unit can propose optimal encryption methods based on the history of past security data. By referring to past security data, optimization of the encryption algorithm becomes possible.

The security unit can improve the accuracy of encryption during data encryption based on the user's health status and lifestyle habits. For example, the security unit improves the accuracy of encryption during data encryption based on the user's health status and lifestyle habits. For instance, the security unit performs data encryption by referencing the user's health checkup results. The security unit can also perform data encryption by considering the user's lifestyle habits (diet, exercise, sleep). Furthermore, the security unit can perform data encryption by considering the user's medical history. By improving the accuracy of encryption based on the user's health status and lifestyle habits, more appropriate data protection becomes possible.

The security unit can estimate the user's emotion and determine the priority of encryption based on the estimated emotion. For example, the security unit estimates the user's emotion and determines the priority of encryption according to the estimated emotion. For instance, if the user is highly motivated, the security unit prioritizes encryption of exercise data. If the user is feeling stressed, the security unit can prioritize encryption of meal data. Furthermore, if the user is relaxed, the security unit can perform encryption by considering overall balance. By determining the priority of encryption according to the user's emotion, more appropriate data protection becomes possible. Emotion estimation is realized, for example, by using an emotion engine or generative AI with emotion estimation functions. Generative AI may include text generation AI (e.g., LLM) or multimodal generative AI, but is not limited to these examples.

The security unit can perform optimal encryption during data encryption by considering the user's geographic location information. For example, the security unit performs optimal encryption during data encryption by considering the user's geographic location information. For instance, the security unit performs encryption by considering security risks in the user's residential area. The security unit can also perform encryption by considering devices that are easily accessible to the user. Furthermore, the security unit can perform encryption by considering legal regulations in the user's residential area. By considering the user's geographic location information, more appropriate data protection becomes possible.

The security unit can analyze the user's social media activity during data encryption and encrypt relevant data. For example, the security unit analyzes the user's social media activity during data encryption and encrypts relevant data. For instance, the security unit refers to data shared by the user on social media. The security unit can also refer to data of influencers followed by the user. Furthermore, the security unit can refer to data from diet communities in which the user participates. By analyzing the user's social media activity, more relevant data protection becomes possible.

The system according to the embodiment is not limited to the examples described above, and various modifications are possible, for example, as follows.

The registration unit can register not only the user's target weight or deadline, but also the user's meal preferences and allergy information. For example, if the user has an allergy to a specific ingredient, registering that information allows the proposed meal plans and recipes to exclude that ingredient. In addition, if the user prefers a specific dietary style (for example, vegetarian or gluten-free), registering that information allows the proposed meal plans and recipes to be adjusted to fit that dietary style. Furthermore, if the user prefers specific ingredients, registering that information allows the proposed meal plans and recipes to actively incorporate those ingredients. As a result, more personalized proposals can be made based on the user's meal preferences and allergy information.

The collection unit can not only include an image recognition unit for analyzing photographs of food consumed, but also record the user's meal times and meal frequency. For example, by recording the user's breakfast, lunch, and dinner times, meal patterns can be identified and appropriate meal timing can be proposed. In addition, by recording how many times the user eats per day, proposals based on meal frequency can be made. Furthermore, by recording the timing and frequency of snacks, snack management and appropriate snack proposals can be provided. As a result, more effective meal management can be achieved based on the user's meal times and frequency.

The collection unit can not only include a cooperation unit for cooperating with a wearable device for collecting exercise data, but also record the types and intensity of the user's exercise. For example, by recording the types of exercise performed by the user (running, walking, cycling, etc.), exercise variations can be identified and appropriate exercise plans can be proposed. In addition, by recording the intensity of exercise performed by the user (light, moderate, high intensity), the effectiveness of exercise can be evaluated and appropriate exercise intensity can be proposed. Furthermore, by recording the time and distance of exercise performed by the user, exercise achievements can be identified and feedback can be provided to maintain motivation. As a result, more effective exercise management can be achieved based on the types and intensity of the user's exercise.

The collection unit is not only provided with a security unit that performs encryption or anonymization of data, but can also record the user's data access history. For example, by recording when and from which device the user accessed the data, it is possible to detect unauthorized access and enhance security. In addition, by recording which data the user accessed, it is possible to grasp the usage status of the data and perform appropriate data management. Furthermore, by recording with whom the user shared the data and the method of sharing, it is possible to manage the data sharing history and strengthen privacy protection. As a result, safer data management can be achieved based on the user's data access history.

The collection unit is not only capable of collecting photographs of food consumed using a smartphone camera, but can also record the user's impressions and satisfaction with meals. For example, by having the user input impressions and satisfaction after a meal, the system can evaluate the meal and reflect this in future meal planning and recipes. In addition, by having the user input preferences for specific ingredients or dishes, the system can grasp the user's dietary preferences and make appropriate suggestions. Furthermore, by having the user input evaluations of the quantity and taste of the meal, feedback can be provided to improve the quality of meals. As a result, more personalized meal suggestions can be made based on the user's impressions and satisfaction with meals.

The analysis unit is not only capable of analyzing collected data using a machine learning algorithm, but can also estimate the user's emotion and adjust the data analysis method based on the estimated emotion. For example, if the user is feeling stressed, the analysis unit can reduce the frequency of data analysis to lessen the user's burden. If the user is highly motivated, the analysis unit can perform detailed data analysis and provide specific feedback to the user. Furthermore, if the user is relaxed, the analysis unit can visually display the results of data analysis in an easy-to-understand manner to promote the user's understanding. Thus, by adjusting the data analysis method according to the user's emotion, more effective data analysis can be achieved.

The registration unit is not only capable of estimating the user's emotion and adjusting the setting of the target weight or deadline based on the estimated emotion, but can also provide messages to enhance motivation according to the user's emotion. For example, if the user is feeling stressed, the registration unit can display encouraging messages to lighten the user's mood. If the user's motivation is low, the registration unit can provide success stories or positive feedback to boost the user's motivation. Furthermore, if the user is approaching the goal, the registration unit can display messages that evoke a sense of achievement to maintain the user's motivation. As a result, by providing messages to enhance motivation according to the user's emotion, more effective goal achievement can be realized.

The registration unit is not only capable of analyzing the user's past diet history and proposing an optimal target setting method, but can also analyze the user's past emotion data and set targets according to changes in emotion. For example, by identifying periods when the user was prone to stress during past dieting, the system can set more moderate goals for those periods. By identifying periods when the user was highly motivated, the system can set stricter goals for those periods. Furthermore, by identifying periods when the user was relaxed, the system can set goals in stages for those periods. As a result, more realistic target setting can be achieved based on the user's past emotion data.

The registration unit is not only capable of performing filtering based on the user's health status or lifestyle habits when setting the target weight or deadline, but can also perform filtering based on the user's emotion. For example, if the user is feeling stressed, the registration unit can propose goals to reduce stress. If the user's motivation is low, the registration unit can propose goals to enhance motivation. Furthermore, if the user is relaxed, the registration unit can propose goals to maintain relaxation. As a result, by setting targets based on the user's emotion, more effective dieting can be achieved.

The registration unit is not only capable of estimating the user's emotion and determining the priority of target setting based on the estimated emotion, but can also provide support for goal achievement according to the user's emotion. For example, if the user is feeling stressed, the registration unit can propose relaxation methods to reduce stress. If the user's motivation is low, the registration unit can propose activities to enhance motivation. Furthermore, if the user is relaxed, the registration unit can provide tips to maintain relaxation. As a result, by providing support for goal achievement according to the user's emotion, more effective dieting can be achieved.

The following is a brief explanation of the processing flow of Example of the Embodiment.
Step 1: The registration unit registers the user's target weight or deadline. For example, the user sets a goal such as “I want to lose 5 kg in 3 months.” This information is input into the system.
Step 2: The collection unit collects data such as the user's weight, exercise data, and photographs of food consumed. For example, the collection unit can have the user take photographs of food consumed using a smartphone camera and collect the data. In addition, the collection unit can cooperate with a wearable device to collect exercise data.
Step 3: The analysis unit analyzes the data collected by the collection unit. For example, the analysis unit can analyze the collected data using a machine learning algorithm.
Step 4: The suggestion unit performs “meal planning,” “recipe creation,” and “exercise plan creation” based on the analysis results obtained by the analysis unit. For example, the suggestion unit can propose meal plans considering calorie restrictions, recipes with balanced nutrition, and effective exercise plans.
Step 5: The modification unit modifies the content proposed by the suggestion unit. For example, each time the user inputs weight, exercise data, or photographs of food consumed, the modification unit analyzes this information and modifies the proposed content.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned registration unit, collection unit, analysis unit, suggestion unit, and modification unit is implemented by at least one of, for example, the smart device 14 and the data processing apparatus 12. For example, the registration unit is implemented by a control unit 46A of the smart device 14 and registers a user's target weight or deadline. The collection unit collects data such as the user's weight, exercise data, and photographs of food consumed using a camera 42 or communication I/F 44 of the smart device 14. The analysis unit is implemented by a specific processing unit 290 of the data processing apparatus 12 and analyzes the collected data. The suggestion unit is implemented by the specific processing unit 290 of the data processing apparatus 12 and performs meal planning, recipe creation, and exercise plan creation based on the analysis results. The modification unit is implemented by the specific processing unit 290 of the data processing apparatus 12 and modifies the proposed content. The correspondence between each unit and the device or control unit is not limited to the examples described above and various modifications are possible.

### Second Embodiment

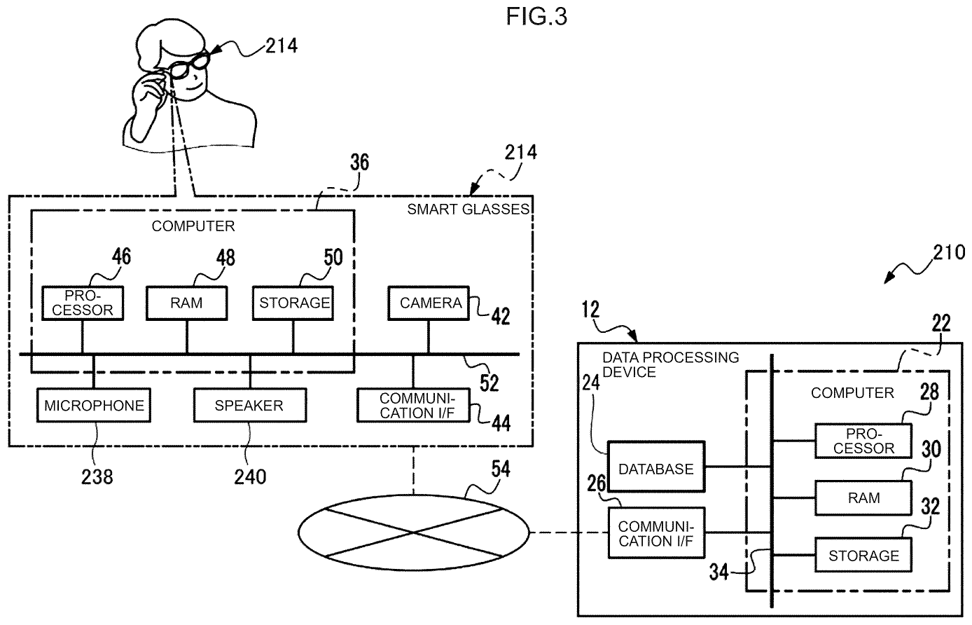

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

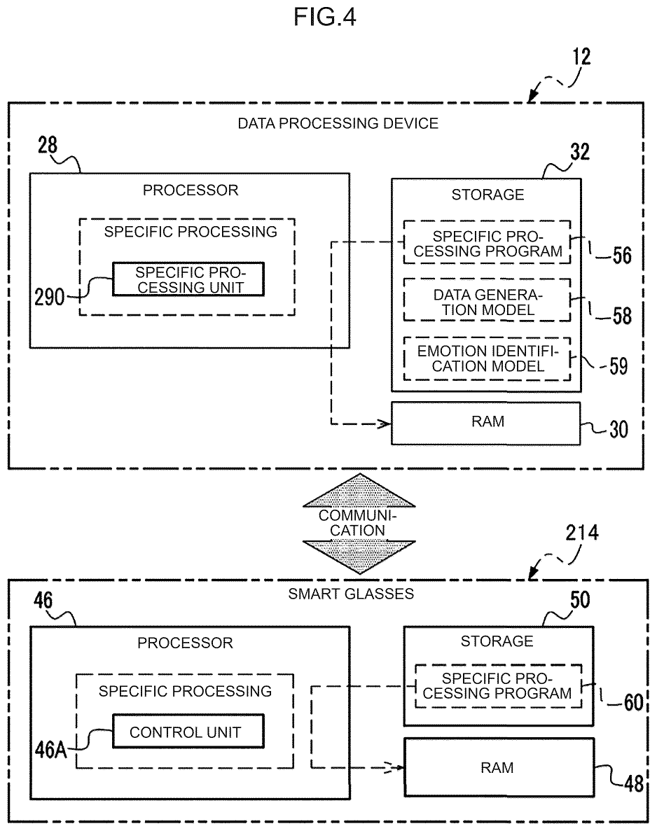

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned registration unit, collection unit, analysis unit, suggestion unit, and modification unit is implemented by at least one of, for example, the smart glasses 214 and the data processing apparatus 12. For example, the registration unit is implemented by a control unit 46A of the smart glasses 214 and registers a user's target weight or deadline. The collection unit collects data such as the user's weight, exercise data, and photographs of food consumed using a camera 42 or communication I/F 44 of the smart glasses 214. The analysis unit is implemented by a specific processing unit 290 of the data processing apparatus 12 and analyzes the collected data. The suggestion unit is implemented by the specific processing unit 290 of the data processing apparatus 12 and performs meal planning, recipe creation, and exercise plan creation based on the analysis results. The modification unit is implemented by the specific processing unit 290 of the data processing apparatus 12 and modifies the proposed content. The correspondence between each unit and the device or control unit is not limited to the examples described above and various modifications are possible.

### Third Embodiment

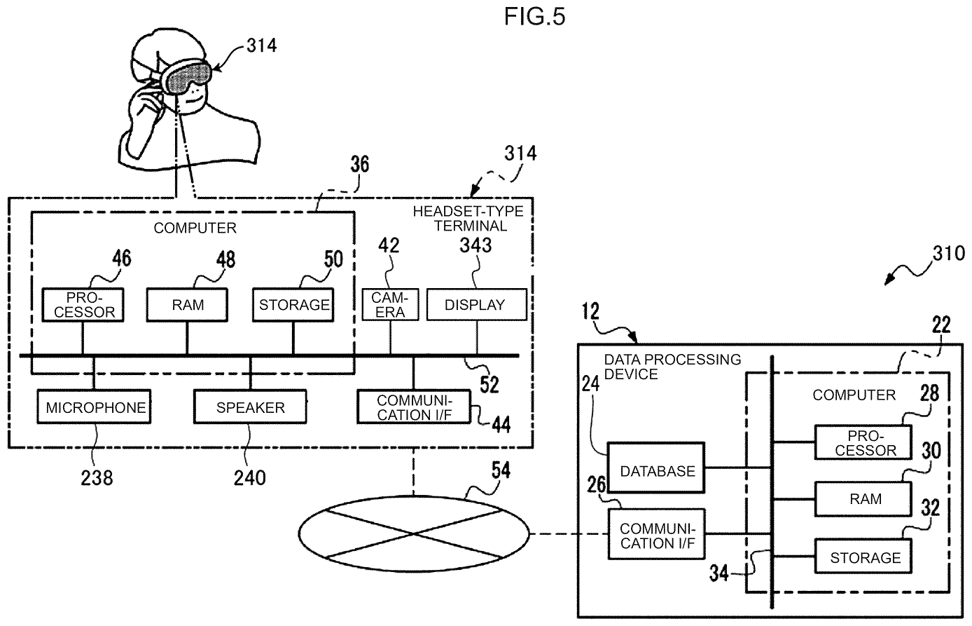

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

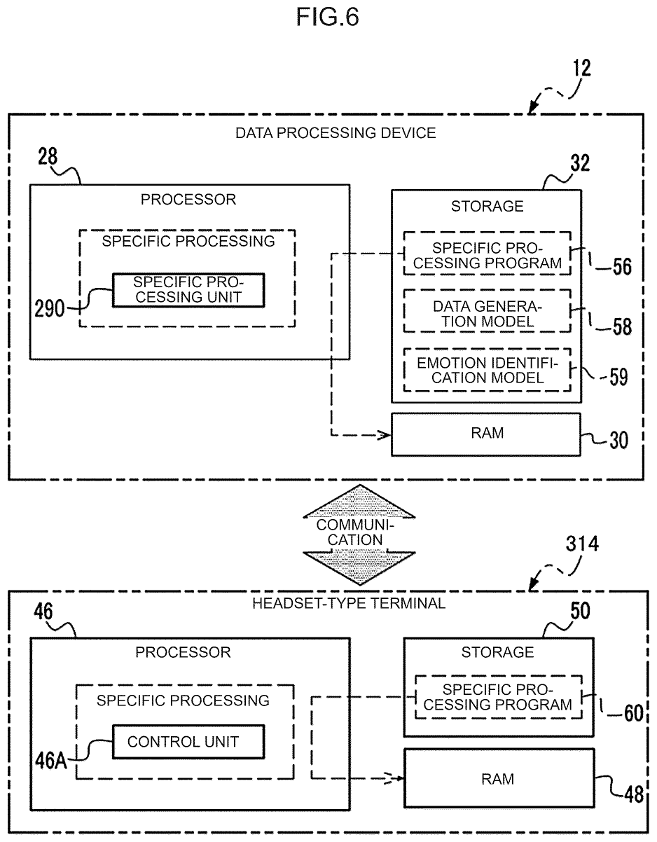

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned registration unit, collection unit, analysis unit, suggestion unit, and modification unit is implemented by at least one of, for example, the headset-type terminal 314 and the data processing apparatus 12. For example, the registration unit is implemented by a control unit 46A of the headset-type terminal 314 and registers a user's target weight or deadline. The collection unit collects data such as the user's weight, exercise data, and photographs of food consumed using a camera 42 or communication I/F 44 of the headset-type terminal 314. The analysis unit is implemented by a specific processing unit 290 of the data processing apparatus 12 and analyzes the collected data. The suggestion unit is implemented by the specific processing unit 290 of the data processing apparatus 12 and performs meal planning, recipe creation, and exercise plan creation based on the analysis results. The modification unit is implemented by the specific processing unit 290 of the data processing apparatus 12 and modifies the proposed content. The correspondence between each unit and the device or control unit is not limited to the examples described above and various modifications are possible.

### Fourth Embodiment

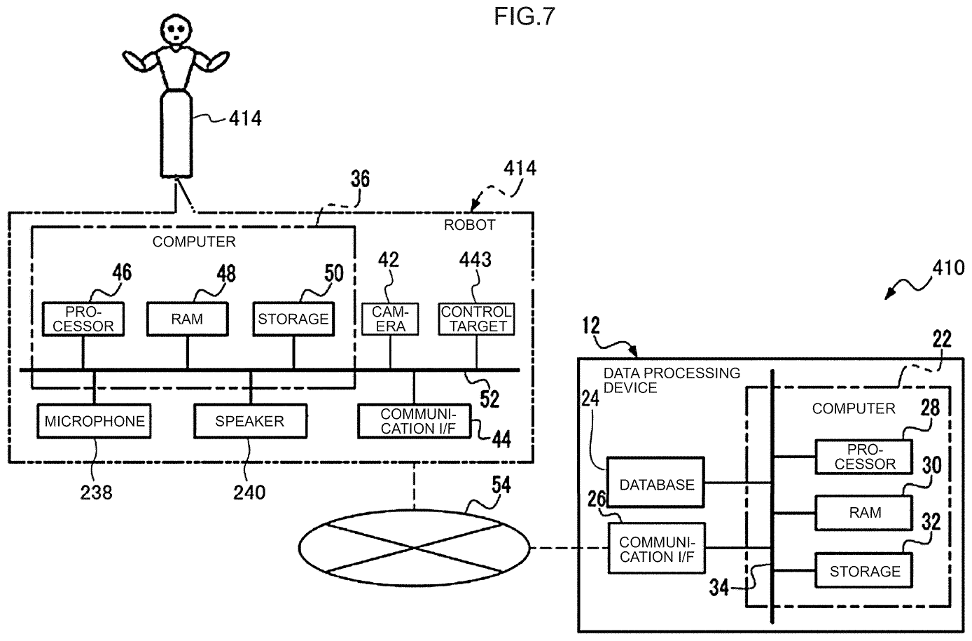

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

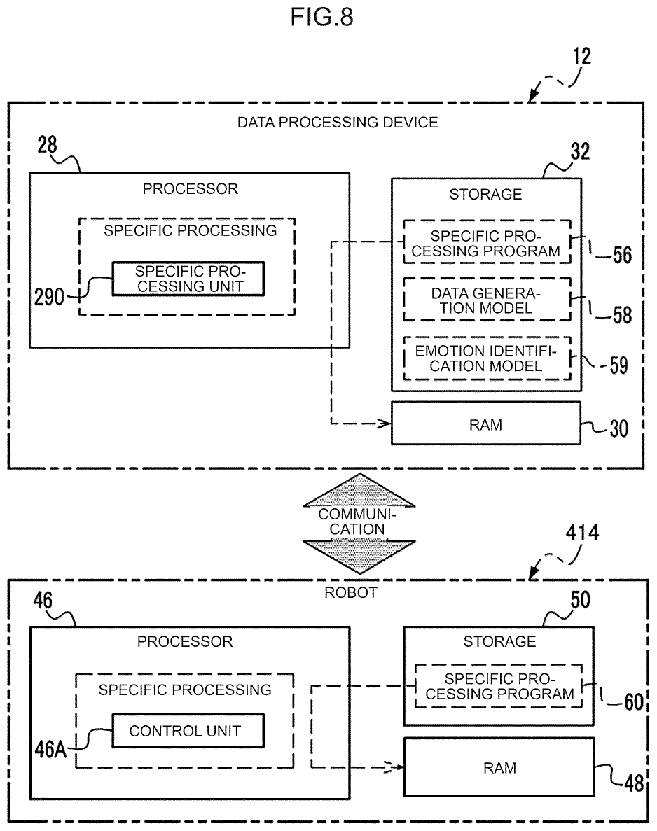

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned registration unit, collection unit, analysis unit, suggestion unit, and modification unit is implemented by at least one of, for example, the robot 414 and the data processing apparatus 12. For example, the registration unit is implemented by a control unit 46A of the robot 414 and registers a user's target weight or deadline. The collection unit collects data such as the user's weight, exercise data, and photographs of food consumed using a camera 42 or communication I/F 44 of the robot 414. The analysis unit is implemented by a specific processing unit 290 of the data processing apparatus 12 and analyzes the collected data. The suggestion unit is implemented by the specific processing unit 290 of the data processing apparatus 12 and performs meal planning, recipe creation, and exercise plan creation based on the analysis results. The modification unit is implemented by the specific processing unit 290 of the data processing apparatus 12 and modifies the proposed content. The correspondence between each unit and the device or control unit is not limited to the examples described above and various modifications are possible.

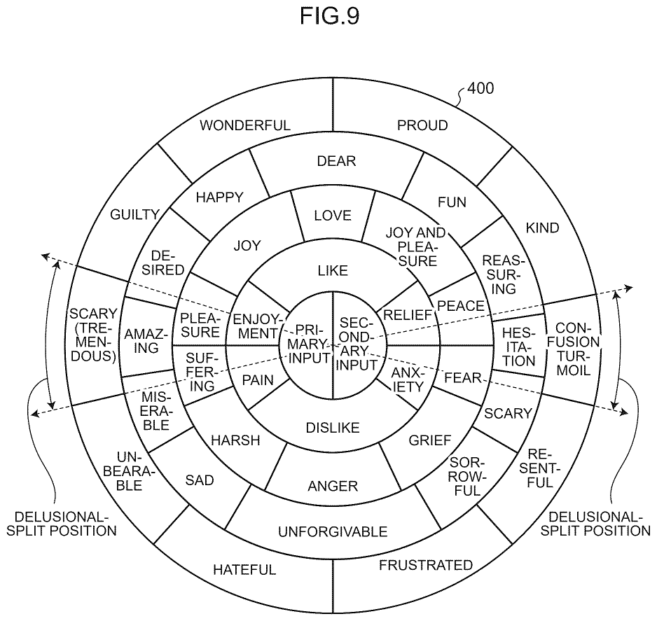

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

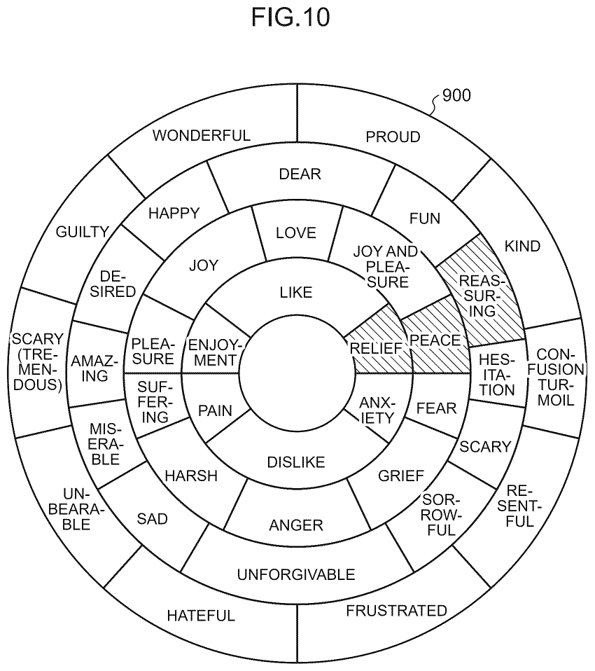

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a registration unit configured to register a user's target weight or deadline; a collection unit configured to collect data including the user's weight, exercise data, and photographs of food consumed; an analysis unit configured to analyze the data collected by the collection unit; a suggestion unit configured to perform meal planning, recipe creation, and exercise plan creation based on the analysis results obtained by the analysis unit; and a modification unit configured to modify the content proposed by the suggestion unit.
(Supplementary Note 2) The system according to Supplementary Note 1, further comprising an image recognition unit configured to analyze photographs of food consumed.
(Supplementary Note 3) The system according to Supplementary Note 1, further comprising a cooperation unit configured to cooperate with a wearable device for collecting exercise data.
(Supplementary Note 4) The system according to Supplementary Note 1, further comprising a security unit configured to perform encryption or anonymization of data.
(Supplementary Note 5) The system according to Supplementary Note 1, wherein the collection unit is configured to collect photographs of food consumed using a smartphone camera.
(Supplementary Note 6) The system according to Supplementary Note 1, wherein the analysis unit is configured to analyze the collected data using a machine learning algorithm.
(Supplementary Note 7) The system according to Supplementary Note 1, wherein the registration unit is configured to estimate a user's emotion and adjust the setting of the target weight or deadline based on the estimated emotion.
(Supplementary Note 8) The system according to Supplementary Note 1, wherein the registration unit is configured to analyze the user's past diet history and propose an optimal target setting method.
(Supplementary Note 9) The system according to Supplementary Note 1, wherein the registration unit is configured to perform filtering based on the user's health status or lifestyle habits when setting the target weight or deadline.
(Supplementary Note 10) The system according to Supplementary Note 1, wherein the registration unit is configured to estimate a user's emotion and determine the priority of target setting based on the estimated emotion.
(Supplementary Note 11) The system according to Supplementary Note 1, wherein the registration unit is configured to preferentially set relevant targets by considering the user's geographic location information when setting the target weight or deadline.
(Supplementary Note 12) The system according to Supplementary Note 1, wherein the registration unit is configured to analyze the user's social media activity and set relevant targets when setting the target weight or deadline.
(Supplementary Note 13) The system according to Supplementary Note 1, wherein the collection unit is configured to estimate a user's emotion and adjust the timing of data collection based on the estimated emotion.
(Supplementary Note 14) The system according to Supplementary Note 1, wherein the collection unit is configured to analyze the user's past data collection history and select an optimal collection method.
(Supplementary Note 15) The system according to Supplementary Note 1, wherein the collection unit is configured to perform filtering based on the user's current living conditions or areas of interest during data collection.
(Supplementary Note 16) The system according to Supplementary Note 1, wherein the collection unit is configured to estimate a user's emotion and determine the priority of data to be collected based on the estimated emotion.
(Supplementary Note 17) The system according to Supplementary Note 1, wherein the collection unit is configured to preferentially collect relevant data by considering the user's geographic location information during data collection.
(Supplementary Note 18) The system according to Supplementary Note 1, wherein the collection unit is configured to analyze the user's social media activity and collect relevant data during data collection.
(Supplementary Note 19) The system according to Supplementary Note 1, wherein the analysis unit is configured to estimate a user's emotion and adjust the data analysis method based on the estimated emotion.
(Supplementary Note 20) The system according to Supplementary Note 1, wherein the analysis unit is configured to optimize the analysis algorithm by referring to past analysis data during data analysis.
(Supplementary Note 21) The system according to Supplementary Note 1, wherein the analysis unit is configured to improve the accuracy of analysis based on the user's health status or lifestyle habits during data analysis.
(Supplementary Note 22) The system according to Supplementary Note 1, wherein the analysis unit is configured to estimate a user's emotion and adjust the display method of analysis results based on the estimated emotion.
(Supplementary Note 23) The system according to Supplementary Note 1, wherein the analysis unit is configured to improve the accuracy of analysis by considering the user's geographic location information during data analysis.
(Supplementary Note 24) The system according to Supplementary Note 1, wherein the analysis unit is configured to analyze relevant data by analyzing the user's social media activity during data analysis.
(Supplementary Note 25) The system according to Supplementary Note 1, wherein the suggestion unit is configured to estimate a user's emotion and adjust the expression method of suggestions based on the estimated emotion.
(Supplementary Note 26) The system according to Supplementary Note 1, wherein the suggestion unit is configured to customize the content of suggestions based on the user's health status or lifestyle habits when making suggestions.
(Supplementary Note 27) The system according to Supplementary Note 1, wherein the suggestion unit is configured to refer to the user's past diet history and make optimal suggestions when making suggestions.
(Supplementary Note 28) The system according to Supplementary Note 1, wherein the suggestion unit is configured to estimate a user's emotion and determine the priority of suggestions based on the estimated emotion.
(Supplementary Note 29) The system according to Supplementary Note 1, wherein the suggestion unit is configured to make optimal suggestions by considering the user's geographic location information when making suggestions.
(Supplementary Note 30) The system according to Supplementary Note 1, wherein the suggestion unit is configured to analyze the user's social media activity and make relevant suggestions when making suggestions.
(Supplementary Note 31) The system according to Supplementary Note 1, wherein the modification unit is configured to estimate a user's emotion and adjust the content of modifications based on the estimated emotion.
(Supplementary Note 32) The system according to Supplementary Note 1, wherein the modification unit is configured to customize the content of modifications based on the user's health status or lifestyle habits when making modifications.
(Supplementary Note 33) The system according to Supplementary Note 1, wherein the modification unit is configured to refer to the user's past diet history and make optimal modifications when making modifications.
(Supplementary Note 34) The system according to Supplementary Note 1, wherein the modification unit is configured to estimate a user's emotion and determine the priority of modifications based on the estimated emotion.
(Supplementary Note 35) The system according to Supplementary Note 1, wherein the modification unit is configured to make optimal modifications by considering the user's geographic location information when making modifications.
(Supplementary Note 36) The system according to Supplementary Note 1, wherein the modification unit is configured to analyze the user's social media activity and make relevant modifications when making modifications.
(Supplementary Note 37) The system according to Supplementary Note 2, wherein the image recognition unit is configured to estimate a user's emotion and adjust the accuracy of image recognition based on the estimated emotion.
(Supplementary Note 38) The system according to Supplementary Note 2, wherein the image recognition unit is configured to optimize the recognition algorithm by referring to past image data during image recognition.
(Supplementary Note 39) The system according to Supplementary Note 2, wherein the image recognition unit is configured to improve the accuracy of recognition based on the user's meal history during image recognition.
(Supplementary Note 40) The system according to Supplementary Note 2, wherein the image recognition unit is configured to estimate a user's emotion and adjust the display method of image recognition results based on the estimated emotion.
(Supplementary Note 41) The system according to Supplementary Note 2, wherein the image recognition unit is configured to improve the accuracy of recognition by considering the user's geographic location information during image recognition.
(Supplementary Note 42) The system according to Supplementary Note 2, wherein the image recognition unit is configured to analyze the user's social media activity and recognize relevant images during image recognition.
(Supplementary Note 43) The system according to Supplementary Note 3, wherein the cooperation unit is configured to estimate a user's emotion and select a cooperation device based on the estimated emotion.
(Supplementary Note 44) The system according to Supplementary Note 3, wherein the cooperation unit is configured to optimize the cooperation algorithm by referring to past cooperation data during cooperation.
(Supplementary Note 45) The system according to Supplementary Note 3, wherein the cooperation unit is configured to customize the content of cooperation based on the user's health status or lifestyle habits during cooperation.
(Supplementary Note 46) The system according to Supplementary Note 3, wherein the cooperation unit is configured to estimate a user's emotion and determine the priority of cooperation based on the estimated emotion.
(Supplementary Note 47) The system according to Supplementary Note 3, wherein the cooperation unit is configured to perform optimal cooperation by considering the user's geographic location information during cooperation.
(Supplementary Note 48) The system according to Supplementary Note 3, wherein the cooperation unit is configured to analyze the user's social media activity and perform relevant cooperation during cooperation.
(Supplementary Note 49) The system according to Supplementary Note 4, wherein the security unit is configured to estimate a user's emotion and adjust the data encryption method based on the estimated emotion.
(Supplementary Note 50) The system according to Supplementary Note 4, wherein the security unit is configured to optimize the encryption algorithm by referring to past security data during data encryption.
(Supplementary Note 51) The system according to Supplementary Note 4, wherein the security unit is configured to improve the accuracy of encryption based on the user's health status or lifestyle habits during data encryption.
(Supplementary Note 52) The system according to Supplementary Note 4, wherein the security unit is configured to estimate a user's emotion and determine the priority of encryption based on the estimated emotion.
(Supplementary Note 53) The system according to Supplementary Note 4, wherein the security unit is configured to perform optimal encryption by considering the user's geographic location information during data encryption.
(Supplementary Note 54) The system according to Supplementary Note 4, wherein the security unit is configured to analyze the user's social media activity and encrypt relevant data during data encryption.

What is claimed is:

1. A system comprising:
circuitry configured to:
receive, from a client terminal via a packet-switched network, target parameter data and input data comprising at least sensor data and image data;
apply a convolutional neural network to the image data to extract a feature vector and generate a classification label;
apply a recurrent neural network to the sensor data to generate a time-series feature vector;
generate, by inputting the feature vector, the classification label, and the time-series feature vector into a data generation model, output plan data; and
generate, by re-inputting updated input data received from the client terminal together with the output plan data into the data generation model, modified plan data reflecting a deviation between the target parameter data and current parameter data derived from the updated input data.

2. The system according to claim 1, wherein the target parameter data comprises a target weight value and a deadline value registered by a user.

3. The system according to claim 1, wherein the sensor data comprises at least one of weight data, exercise data including step count and calories burned, and heart rate data collected as a time-series vector.

4. The system according to claim 1, wherein the image data comprises an RGB image tensor captured by a camera of the client terminal, and wherein the classification label identifies a food item depicted in the image data.

5. The system according to claim 1, wherein the convolutional neural network comprises an object detection head configured to detect a plurality of regions in the image data as bounding boxes, and a classification head configured to generate a respective classification label for each detected region.

6. The system according to claim 5, wherein the circuitry is further configured to input the classification label for each detected region into a multilayer perceptron to generate an estimated nutrient value for each detected region.

7. The system according to claim 1, wherein the recurrent neural network comprises a long short-term memory network configured to extract an activity pattern from the sensor data and generate an intensity score.

8. The system according to claim 1, wherein the circuitry is further configured to:
input at least one of text data, audio data, and image data received from the client terminal into an emotion identification model; and
generate an emotion score indicating an emotional state of a user,
wherein the circuitry adjusts at least one of the target parameter data and the output plan data based on the emotion score.

9. The system according to claim 8, wherein the emotion identification model comprises a multimodal model combining a transformer-based language model, a convolutional neural network for image classification, and a recurrent neural network for audio classification.

10. The system according to claim 8, wherein the circuitry determines an emotion value corresponding to a position on an emotion map in which a plurality of emotions are arranged concentrically, and adjusts the output plan data based on the emotion value.

11. The system according to claim 1, wherein the circuitry is further configured to receive the sensor data from a wearable device communicatively coupled to the client terminal via a wireless communication protocol.

12. The system according to claim 1, wherein the circuitry is further configured to encrypt the input data using at least one of symmetric key encryption and public key encryption before storing the input data.

13. The system according to claim 1, wherein the data generation model comprises a reinforcement learning model configured to generate the output plan data by optimizing a reward function based on a goal achievement rate derived from the target parameter data and the current parameter data.

14. The system according to claim 1, wherein the circuitry is further configured to:
retrieve historical input data associated with a user from a database; and
apply a machine learning model to the historical input data to generate a recommended target parameter, wherein the target parameter data is adjusted based on the recommended target parameter.

15. The system according to claim 1, wherein the circuitry is further configured to preprocess the image data by performing at least one of resizing, noise removal, and color space conversion before applying the convolutional neural network.

16. The system according to claim 1, wherein the circuitry is further configured to:
compute a progress score by comparing the current parameter data with the target parameter data; and
select, based on the progress score exceeding or falling below a threshold, a modification strategy for generating the modified plan data.

17. The system according to claim 1, wherein the client terminal comprises at least one of a smart device, smart glasses, a headset-type terminal, and a robot.

18. A system comprising:
a communication interface configured to communicate with a client terminal via a packet-switched network;
a processor;
a random-access memory;
a memory storing a convolutional neural network, a recurrent neural network, a data generation model, and an emotion identification model; and
circuitry configured to:
receive, via the communication interface, target parameter data and input data comprising sensor data and image data from the client terminal;
apply the convolutional neural network to the image data to extract a feature vector and generate a classification label;
apply the recurrent neural network to the sensor data to generate a time-series feature vector;
generate, by inputting the feature vector, the classification label, and the time-series feature vector into the data generation model, output plan data;
input at least one of text data, audio data, and image data received from the client terminal into the emotion identification model to generate an emotion score; and
generate, by re-inputting updated input data received from the client terminal together with the output plan data and the emotion score into the data generation model, modified plan data reflecting a deviation between the target parameter data and current parameter data derived from the updated input data.

19. The system according to claim 18, wherein the emotion identification model comprises a multimodal model combining a transformer-based language model configured to process the text data, a convolutional neural network configured to process the image data, and a recurrent neural network configured to process the audio data, and wherein the emotion score comprises a plurality of emotion dimension values corresponding to positions on an emotion map.

20. A method performed by circuitry of a system, the method comprising:
receiving, from a client terminal via a packet-switched network, target parameter data and input data comprising at least sensor data and image data;
applying a convolutional neural network to the image data to extract a feature vector and generate a classification label;
applying a recurrent neural network to the sensor data to generate a time-series feature vector;
generating, by inputting the feature vector, the classification label, and the time-series feature vector into a data generation model, output plan data; and
generating, by re-inputting updated input data received from the client terminal together with the output plan data into the data generation model, modified plan data reflecting a deviation between the target parameter data and current parameter data derived from the updated input data.
